(19) United States
(12) Patent Application Publication
(10) Pub. No.: US 2020/0138680 A1
(43) Pub. Date: May 7, 2020

(54) GEL-TYPE COSMETIC COMPOSITION

(71) Applicant: L'OREAL, Paris (FR)

(72) Inventors: Véronique FERRARI, Maisons-Alfort (FR); Elodie VALVERDE, Paris (FR)

(73) Assignee: L'OREAL, Paris (FR)

(21) Appl. No.: 16/729,918

(22) Filed: Dec. 30, 2019

Related U.S. Application Data
(63) Continuation of application No. 14/770,154, filed on Aug. 25, 2015.

(30) Foreign Application Priority Data
Feb. 25, 2013 (FR) 13 00429

(51) Int. Cl. A61K 8/04; A61Q 19/00; A61K 8/81; A61K 8/891; A61K 8/88; A61Q 1/12; A61Q 1/10; A61Q 1/06; A61Q 1/02; A61K 8/84; A61K 8/898

(52) U.S. Cl. CPC A61K 8/042; A61Q 19/007; A61K 8/8152; A61K 8/891; A61K 8/88; A61Q 1/12; A61K 2800/882; A61Q 1/06; A61Q 1/02; A61K 8/84; A61Q 19/00; A61K 8/8158; A61K 8/898; A61Q 1/10

(57) ABSTRACT

A cosmetic gel-gel type composition for making up and/or caring for keratin materials, includes (i) at least one aqueous phase gelled with at least one hydrophilic gelling agent selected from the group consisting of copolymers of (1) 2-acrylamido-2-methylpropanesulfonic acid and (2) hydroxyethyl acrylate; and (ii) at least one oily phase gelled with at least one lipophilic gelling agent selected from the group consisting of hydrocarbon-based polyamides, silicone polyamides, and mixtures thereof. Either (a) the at least one hydrophilic gelling agent is a copolymer of 2-acrylamido-2-methylpropanesulfonic acid and of hydroxyethyl acrylate, and the at least one lipophilic gelling agent is ethylenediamine/stearyl dimer dilinoleate copolymer, or (b) the at least one hydrophilic gelling agent is a copolymer of 2-acrylamido-2-methylpropanesulfonic acid and of hydroxyethyl acrylate, and the at least one lipophilic gelling agent is Nylon-611/dimethicone copolymer.

The present invention is directed towards proposing for the field of caring for and making up keratin materials, especially the skin and/or the lips, and in particular the skin, a novel galenical form that is most particularly advantageous with regard to its technical performance and the sensations it affords the user during its application, in particular to the skin.

The term “keratin materials” especially means the skin, the lips and/or the eyelashes, in particular the skin and/or the lips, and preferably the skin.

Conventionally, a cosmetic composition formulator uses emulsified systems combining an aqueous phase for freshness and an oily phase for comfort. The strong point of these systems is also that they allow the combination, within the same composition, of cosmetic ingredients or active agents that have different affinities with respect to these two aqueous and oily phases.

Unfortunately, these emulsified systems do not lend themselves to rapid and easy production of an infinite range of compositions. Thus, for a given emulsified system, it often proves complicated to functionalize the formulation by adding, for example, an antisun product, certain active agents, pigments, polymers, fragrances or fillers, etc. without impairing the stability, the sensory properties and the quality of the film deposited on the keratin materials and especially the skin. The formulation then needs to be readjusted. It is also difficult to reconcile, within the same composition, opposing technical performance qualities, for instance mattness (which may make the skin dry) and moisturization (which may make the skin shiny).

Furthermore, emulsified systems do not lend themselves to the formulation of all the ingredients or active agents liable to be considered in the field of care and/or makeup, or even to the formulation of high contents of certain cosmetic ingredients or active agents. Non-compliance with these incompatibilities has the consequence of destabilizing the emulsified architecture, which then, inter alia, undergoes demixing.

Finally, these emulsified systems do not lend themselves to rapid and easy production of an infinite range of textures.

Moreover, in the case of making up the complexion, the preferred emulsifying systems are mainly reverse emulsions with regard to the good level of coverage and the homogeneous appearance they afford when compared with direct emulsions. On the other hand, their weak point is a high greasy and tacky sensation, and thus a lack of lightness as regards the textures obtained.

Galenical formulations of gel/gel type partially meet these expectations (Almeida et al., Pharmaceutical Development and Technology, 2008, 13:487, tables 1 and 2, page 488; WO 99/65455; PI 0405758-9; WO 99/62497; JP 2005-112834 and WO 2008/081175). Formulations of this type combine a gelled aqueous phase with a gelled oily phase. In fact, these gel/gel formulations were essentially proposed as an advantageous alternative to emulsified systems on the grounds that they make it possible to dispense with the use of the surfactants required for the stability and texturization of emulsions. Unfortunately, besides this advantage, the gel/gel formulations described hitherto do not essentially reveal any novel or improved technical performance qualities.

It therefore remains difficult for a person skilled in the art to propose homogeneous compositions that are capable of affording an immediate visual result on the skin with a light sensation on application, this expected immediate result preferentially being good coverage of colour imperfections and/or of relief imperfections, without, however, marking them. It is therefore necessary to find novel systems for distributing on the skin components such as water, fatty substances and solid particles.

These novel architectures must be entirely satisfactory to users as regards the sensation afforded, but must also be capable of affording improved cosmetic properties, or must even have an increased number of technical performance qualities such as freshness, lightness, emollience, comfort, coverage of imperfections, colour, unifying aspect, lightening, etc., and, on the other hand, must be free of the known side effects of oily and aqueous phases such as, respectively, a greasy feel, a tacky feel, a feeling of lack of glidance or alternatively a feeling of dragging on application.

The inventors have now found, unexpectedly, that such an objective can be achieved via the choice of a system of specific hydrophilic gelling agent(s)/lipophilic gelling agent(s) for the preparation of a cosmetic composition of the type such as a bi-continuous but on the other hand macroscopically homogeneous system which has a large number of technical performance qualities and which furthermore has optimized effects.

More precisely, the inventors have found that the choice of a system of specific hydrophilic gelling agent(s)/lipophilic gelling agent(s) makes it possible, contrary to all expectation, to combine in a single composition a significant number of technical performance qualities, with the intensity of each performance quality advantageously not being attenuated by the manifestation of other associated performance qualities, or even being, for certain performance qualities, stimulated.

Thus, according to one of its aspects, the present invention relates to a cosmetic composition for making up and/or caring for keratin materials, in particular the skin and/or the lips, comprising:
at least one aqueous phase gelled with at least one hydrophilic gelling agent; and
at least one oily phase gelled with at least one hydrogen bonding polymer; the said phases forming therein a macroscopically homogeneous mixture.

According to one embodiment variant, a composition according to the invention consists of an aqueous phase gelled with at least one hydrophilic gelling agent and an oily phase gelled with at least one hydrogen bonding polymer.

According to a preferred variant, a composition according to the invention also contains at least one dyestuff.

This dyestuff may be chosen from pigments, and water-soluble or liposoluble dyestuffs, especially as detailed below.

In particular, the dyestuffs are pigments.

According to an advantageous embodiment variant, the dyestuff is conveyed at least in the gelled oily phase.

As stated above, the inventors have found, contrary to all expectation, that the choice of particular hydrophilic gelling agent(s)/lipophilic gelling agent(s) couples for texturing a composition of gel/gel type makes it possible to significantly improve certain technical performance qualities, and to dispense with certain adverse effects inherent in the gelling agents under consideration, or even to reconcile within this composition properties which it was hitherto difficult to make coexist. Furthermore, as emerges from the examples below, the present invention moreover makes it possible, unexpectedly, to optimize some of the expected technical performance qualities.

The inventors have also found, surprisingly, that the soft-focus performance quality of a composition according to the invention comprising aqueous and oily phases gelled, respectively, with a polymeric or particulate gelling agent with a soft-focus effect proves to be significantly improved. The gain in soft-focus effect proves to be greater than the sum of the respective optical effects of each of the two gelled phases in each of the two compositions. There is manifestly synergism.

Besides the abovementioned unexpected advantages, the gelling system under consideration according to the invention affords a texture that is sufficiently thickened to be compatible with the formulation of a very wide diversity of ingredients or active agents. It combines in a single formulation a large number of functional active agents or ingredients (fillers, pigments, etc.).

The compositions according to the invention also prove to be very stable and not subject to syneresis.

According to another of its aspects, a subject of the invention is also a process for preparing a cosmetic composition for making up and/or caring for keratin materials, in particular the skin and/or the lips, comprising at least one step of mixing:
at least one aqueous phase gelled with at least one hydrophilic gelling agent; and
at least one oily phase gelled with at least one hydrogen bonding polymer; under conditions suitable for obtaining a macroscopically homogeneous mixture.

According to one embodiment variant, this process may advantageously comprise a step of mixing at least three or even more gelled phases.

For obvious reasons, the number of gelled aqueous phases and of gelled oily phases to be considered for forming a composition according to the invention may range for each of the two types of phase beyond two. It is especially conditioned by the number of expected technical performance qualities.

For example, this process may use a single gelled aqueous phase and two oily phases gelled with different lipophilic gelling agents.

Conversely, this process may also use a single gelled oily phase and two aqueous phases gelled with different hydrophilic gelling agents.

For example, the phases having the same architecture, namely aqueous or oily, may be precombined to form a premix, and it is this premix which is placed in contact with the phase or even with a premix of several phases having the other architecture.

The corresponding aqueous and oily gels may be prepared separately then mixed together without heating, without requiring the necessary presence of surfactants in order to achieve the desired architecture. Thus, in addition to the advantages mentioned above, the claimed compositions may be readily prepared at reduced cost.

Advantageously, the mixing of the phases may be performed at room temperature.

However, the process of the invention may comprise, if necessary, a step of heating the mixture.

The process according to the invention thus offers the formulator a simple and rapid means for gaining access to a multitude of cosmetic compositions having common performance qualities but also performance qualities that are specific to each of its compositions.

The present invention also gives the user access to this faculty of mixing at least two phases of the same architecture with at least one phase of different architecture via the provision of a cosmetic kit for making up and/or caring for keratin materials.

Thus, according to another of its aspects, the present invention relates to a cosmetic kit for making up and/or caring for keratin materials, in particular the skin and/or the lips, comprising, in separate containers, at least one aqueous phase gelled with at least one hydrophilic gelling agent; and at least one oily phase gelled with at least one hydrogen bonding polymer; and also to instructions for using the extemporaneous mixtures.

According to yet another of its aspects, the present invention relates to a device for making up and/or caring for keratin materials, in particular the skin and/or the lips, comprising at least:
two separate containers containing, respectively, at least one aqueous phase gelled with at least one hydrophilic gelling agent and at least one oily phase gelled with at least one hydrogen bonding polymer;
a distinct chamber for mixing the said containers, comprising an aperture configured to allow the introduction of the said phases to be mixed; and
a means for distributing a macroscopically homogeneous mixture of the two phases.

According to an advantageous variant, the kits and devices according to the invention contain at least two, or even more, different gelled phases for each of the two types of aqueous and oily architecture.

According to a particular embodiment, the representative gelled phases of the same type of architecture are gelled with a different gelling agent.

Multi-phase formulations of “patchwork” type may thus be developed.

According to another particular embodiment, the representative gelled phases of the same type of architecture are different as regards their optical properties. For example, the kit or device may propose two oily gelled phases textured by the same oily gelling agent, but one containing dyestuffs and the other not. The user thus has the possibility of exploiting or not exploiting makeup performance quality in addition to the other performance qualities.

A kit or device according to the invention also allows the user to modify the intensity of the colour effect by adjusting the proportion of the coloured gelled phase to be mixed.

Thus, the kits and devices according to the invention are particularly advantageous in so far as they afford the user the possibility of adjusting at will, by means of the choice of the gelled phases representative of the two types of oily and aqueous architecture, the desired makeup performance qualities, while at the same time ensuring convenience and ease of use.

The present invention especially makes it possible to afford the user a wider makeup range and also to give the makeup operation an appealing fun aspect. Moreover, the fact that the mixing of the phases may be performed at room temperature is of manifest interest as regards the convenience and thus gives satisfaction as regards the simplicity of use.

According to another of its aspects, a subject of the invention is also a process, especially a cosmetic process, for making up and/or caring for a keratin material, in particular the skin and/or the lips, comprising at least one step that consists in applying to the said keratin material a composition in accordance with the invention.

According to yet another of its aspects, the present invention relates to a process, especially a cosmetic process, for caring for and/or making up a keratin material, in particular the skin and/or the lips, comprising at least the application to the said material of a composition, in particular a macroscopically homogeneous composition obtained by extemporaneous mixing, before application or at the time of application to the said keratin material, of at least one aqueous phase gelled with at least one hydrophilic gelling agent, and of at least one oily phase gelled with at least one hydrogen bonding polymer.

### Cosmetic Composition

Firstly, it is important to note that a composition according to the invention is different from an emulsion.

An emulsion generally consists of an oily liquid phase and an aqueous liquid phase. It is a dispersion of droplets of one of the two liquid phases in the other. The size of the droplets forming the dispersed phase of the emulsion is typically about a micrometer (0.1 to 100 μm). Furthermore, an emulsion requires the presence of a surfactant or of a silicone emulsifier to ensure its stability over time.

In contrast, a composition according to the invention consists of a macroscopically homogeneous mixture of two immiscible gelled phases. These two phases both have a gel-type texture. This texture is especially reflected visually by a consistent, creamy appearance.

The term “macroscopically homogeneous mixture” means a mixture in which each of the gelled phases cannot be individualized with the naked eye.

More precisely, in a composition according to the invention, the gelled aqueous phase and the gelled oily phase interpenetrate and thus form a stable, and consistent product. This consistency is achieved by mixing interpenetrated oily and aqueous gelled macrodomains. These interpenetrated macrodomains are not measurable objects. Thus, by microscope, the composition according to the invention is very different from an emulsion. It cannot be characterized either as having a “sense”, i.e. an O/W or W/O sense.

Thus, a composition according to the invention has a gel-type consistency. Furthermore, the stability of the composition is long-lasting without surfactant. Consequently, a cosmetic composition according to the invention does not require any surfactant or silicone emulsifier to ensure its stability over time.

It is known from the state of the art to observe the intimate nature of the mixture of the aqueous and oily gels in a gel-type composition, for example, by introducing a dye substance into either the oily or aqueous gel phases before forming the gel-type composition. On visual inspection, the dye is seen to be uniformly dispersed, even though the dye is present in only one of the oily gel or aqueous gel. Indeed, if two different dyes of different colours are introduced into the oily and aqueous phases, respectively, before forming the gel-type composition, both colours can be observed uniformly dispersed throughout the gel-type composition. This is in contrast to an emulsion wherein if a dye that is either water-soluble or oil-soluble is introduced into the aqueous or oily phases, respectively, before forming an emulsion, only the colour of the dye in the external phase will be observed (Remington: The Science and Practice of Pharmacy, 19th Edition (1995) Chapter 21, page 282).

It is also known to distinguish a gel-type composition from an emulsion by performing a “drop test”. This test consists to demonstrate the bi-continous nature of a gel-type composition. Indeed, as mentioned above, the composition's consistency is achieved by interpenetrating oily and aqueous gelled domains. Therefore, the bi-continous nature of a gel-type composition can be highlighted by a simple test with respectively hydrophilic and hydrophobic solvents. This test consists to deposit, on the one hand, a droplet of a hydrophilic solvent on a first sample of the tested composition, and, on the other hand, a droplet of hydrophobic solvent on a second sample of the same tested composition, and to analyze the behavior of both droplets of solvents. In the case of an O/W emulsion, a droplet of hydrophilic solvent diffuses in the sample and a droplet of hydrophobic solvent remains at the sample surface. In the case of a W/O emulsion, a droplet of hydrophilic solvent remains at the sample surface and a droplet of hydrophobic solvent diffuses throughout sample. Finally, in the case of a gel-type composition (bi-continuous system), the hydrophilic and hydrophobic droplets diffuse in the entire sample.

In particular, in the case of the present invention, the test which will be privileged for distinguishing a gel-type composition from an emulsion consists in a dilution test. Indeed, in a gel-type composition, the gelled aqueous domains and gel oily domains interpenetrate and form a stable and consistent product, whose dilution behavior in water and oil is different of emulsion's behavior. Therefore, the dilution behavior of a gel-type composition (bi-continuous system) can be compared to emulsions.

More specifically, the dilution test consists to put 40 g of product plus 160 g of dilution solvent (water or oil) in a 30 ml plastic beaker. The dilution is performed under controlled agitation to avoid any phenomenon of emulsification. In particular, it is done using a planetary mixer: Speed Mixer™ DAC400FVZ. The Speed Mixer is set to 1500 rpm for 4 minutes. Finally, observation of resulting sample is made with a light microscope at a magnification of ×100 (×10×10). It may be noticed that oils like Parleam® and Xiameter PMX-200 Silicone Fluid 5CS® from Dow Corning are convenient as dilution solvents.

In the case of a gel-type composition (bi-continuous system), when diluted either in oil or water, a heterogeneous aspect is always observed. When a gel-type composition (bi-continuous system) is diluted with water, one will observe lumps of oily gel in suspension and when a gel-type composition (bi-continuous system) is diluted with oil, one will observe lumps of aqueous gel in suspension.

On the contrary, upon dilution, emulsions display a different behavior. An O/W emulsion when it is diluted with an aqueous solvent will gradually thin up without presenting a heterogeneous and lumpy aspect. This same O/W emulsion when diluted with oil will present a heterogeneous appearance (lumps of O/W emulsion suspended in oil). A W/O emulsion when diluted with an aqueous solvent will present a heterogeneous appearance (lumps of W/O emulsion is suspended in the water). This same W/O emulsion when diluted with oil will gradually thin up without presenting a heterogeneous and lumpy aspect.

In general, the aqueous gelled phase and the oily gelled phase forming a composition according to the invention are present therein in a weight ratio ranging from 95/5 to 5/95. More preferentially, the aqueous phase and the oily phase are present in a weight ratio ranging from 30/70 to 80/20.

The ratio between the two gelled phases is adjusted according to the desired cosmetic properties.

Thus, in the case of a composition intended for making up the skin and especially the face, it is advantageous to favour an aqueous phase/oily phase weight ratio greater than 1, especially ranging from 60/40 to 90/10, preferably ranging from 60/40 to 80/20, preferably from 60/40 to 70/30, and more preferably to favour an aqueous phase/oily phase weight ratio of 60/40 or 70/30.

These preferred ratios are particularly advantageous for obtaining fresh and light compositions.

Advantageously, a composition according to the invention is in the form of a creamy gel with a minimum stress below which it does not flow unless it has been subjected to an external mechanical stress.

As emerges from the text hereinbelow, a composition according to the invention may have a minimum threshold stress of 1.5 Pa and in particular greater than 10 Pa.

It may also advantageously have a stiffness modulus G* at least equal to 400 Pa and preferably greater than 1000 Pa.

According to an advantageous embodiment variant, the gelled phases under consideration to form a composition according to the invention may have, respectively, a threshold stress of greater than 1.5 Pa and preferably greater than 10 Pa.

Characterization of the threshold stresses is performed by oscillating rheology measurements. A method is proposed in the examples section of the present text.

In general, the corresponding measurements are taken at 25° C. using a Haake RS600 imposed-stress rheometer equipped with a plate-plate measuring body (60 mm diameter) fitted with an anti-evaporation device (bell jar). For each measurement, the sample is placed delicately in position and the measurements start 5 minutes after placing the sample in the air gap (2 mm). The tested composition is then subjected to a stress ramp from 10⁻² to 10³ Pa at a set frequency of 1 Hz.

A composition according to the invention may also have a certain elasticity. This elasticity may be characterized by a stiffness modulus G* which, under this minimum stress threshold, may be at least equal to 400 Pa and preferably greater than 1000 Pa. The value G* of a composition may be obtained by subjecting the composition under consideration to a stress ramp from 10⁻² to 10³ Pa at a set frequency of 1 Hz.

### Hydrophilic Gelling Agent

For the purposes of the present invention, the term “hydrophilic gelling agent” means a compound that is capable of gelling the aqueous phase of the compositions according to the invention.

The gelling agent is hydrophilic and is thus present in the aqueous phase of the composition.

The gelling agent may be water-soluble or water-dispersible.

As stated above, the aqueous phase of a composition according to the invention is gelled with at least one hydrophilic gelling agent.

The hydrophilic gelling agent is chosen from synthetic polymeric gelling agents, polymeric gelling agents that are natural or of natural origin, mixed silicates and fumed silicas, and mixtures thereof.

I. Polymeric Telling Agents that are Natural or of Natural Origin

The polymeric hydrophilic gelling agents that are suitable for use in the invention may be natural or of natural origin.

For the purposes of the invention, the expression “of natural origin” is intended to denote polymeric gelling agents that are obtained by modification of natural polymeric gelling agents.

These gelling agents may be particulate or non-particulate.

More precisely, these gelling agents fall within the category of polysaccharides.

In general, polysaccharides may be divided into several categories.

Thus, polysaccharides that are suitable for use in the invention may be homopolysaccharides such as fructans, glucans, galactans and mannans or heteropolysaccharides such as hemicellulose.

Similarly, they may be linear polysaccharides such as pullulan or branched polysaccharides such as gum arabic and amylopectin, or mixed polysaccharides such as starch.

More particularly, the polysaccharides that are suitable for use in the invention may be distinguished according to whether or not they are starchy.

### Starchy Polysaccharides

Representatives of this category that may most particularly be mentioned include native starches, modified starches and particulate starches.

Native Starches

The starches that may be used in the present invention are more particularly macromolecules in the form of polymers consisting of elemental units which are anhydroglucose (dextrose) units, linked via α(1,4) bonds, of chemical formula (C₆H₁₀O₅)ₙ. The number of these units and their assembly make it possible to distinguish amylose, which is a molecule formed from about 600 to 1000 linearly linked glucose molecules, and amylopectin, which is a polymer that is branched every 25 glucose residues approximately (α(1,6) bond). The total chain may contain between 10 000 and 100 000 glucose residues.

Starch is described in particular in Kirk-Othmer's Encyclopaedia of Chemical Technology, 3rd edition, volume 21, pages 492-507, Wiley Interscience, 1983.

The relative proportions of amylose and of amylopectin, and their degree of polymerization, vary as a function of the botanical origin of the starches. On average, a sample of native starch consists of about 25% amylose and 75% amylopectin.

Occasionally, phytoglycogen is present (between 0% and 20% of starch), this molecule being an analogue of amylopectin but branched every 10 to 15 glucose residues.

Starch may be in the form of semi-crystalline granules: amylopectin is organized in leaflets, amylose forms an amorphous zone that is less well organized between the various leaflets.

Amylose self-organizes in a right-handed helix with six glucoses per turn. It dissociates into glucose which may be assimilated under the action of enzymes, amylases, all the more readily if it is in the form of amylopectin. Specifically, the helix formation does not favour the accessibility of starch to enzymes.

Starches are generally in the form of a white powder, which is insoluble in cold water, whose elemental particle size ranges from 3 to 100 microns.

By treating it with hot water, starch paste is obtained. It is used in industry for its thickening and gelling properties.

The botanical origin of the starch molecules used in the present invention may be cereals or tubers. Thus, the starches are chosen, for example, from corn starch, rice starch, cassava starch, tapioca starch, barley starch, potato starch, wheat starch, sorghum starch and pea starch.

Native starches are represented, for example, by the products sold under the names C*Amilogel™, Cargill Gel™, C* Gel™, Cargill Gum™, DryGel™ and C*Pharm Gel™ by the company Cargill, under the name Amidon de mals by the company Roquette, and under the name Pure Tapioca by the company National Starch.

Modified Starches

The modified starches used in the composition of the invention may be modified via one or more of the following reactions: pregelatinization, degradation (acid hydrolysis, oxidation or dextrinization), substitution (esterification or etherification), crosslinking (esterification), bleaching.

More particularly, these reactions may be performed in the following manner:
pregelatinization by splitting the starch granules (for example drying and cooking in a drying drum);
acid hydrolysis giving rise to very rapid retrogradation on cooling;
oxidation with strong oxidizing agents (alkaline medium, in the presence of sodium hypochlorite NaOCl, for example) leading to depolymerization of the starch molecule and to the introduction of carboxyl groups into the starch molecule (mainly oxidation of the C₆ hydroxyl group);
dextrinization in acidic medium at high temperature (hydrolysis followed by repolymerization);
crosslinking with functional agents capable of reacting with the hydroxyl groups of starch molecules which will thus be linked together (for example with glyceryl and/or phosphate groups);
esterification in alkaline medium for the grafting of functional groups, especially C₁-C₆ acyl (acetyl), C₁-C₆ hydroxyalkyl (hydroxyethyl or hydroxypropyl), carboxymethyl or octenylsuccinic.

Monostarch phosphates (of the type St-O—PO—(OX)₂), distarch phosphates (of the type St-O—PO—(OX)—O-St) or even tristarch phosphates (of the type St-O—PO—(O-St)₂) or mixtures thereof may especially be obtained by crosslinking with phosphorus compounds.

X especially denotes alkali metals (for example sodium or potassium), alkaline-earth metals (for example calcium or magnesium), ammonium salts, amine salts, for instance those of monoethanolamine, diethanolamine, triethanolamine, 3-amino-1,2-propanediol, or ammonium salts derived from basic amino acids such as lysine, arginine, sarcosine, ornithine or citrulline.

The phosphorus compounds may be, for example, sodium tripolyphosphate, sodium orthophosphate, phosphorus oxychloride or sodium trimetaphosphate.

According to the invention, it is also possible to use amphoteric starches, these amphoteric starches containing one or more anionic groups and one or more cationic groups. The anionic and cationic groups may be linked to the same reactive site of the starch molecule or to different reactive sites; they are preferably linked to the same reactive site. The anionic groups may be of carboxylic, phosphate or sulfate type, preferably carboxylic. The cationic groups may be of primary, secondary, tertiary or quaternary amine type.

The amphoteric starches are especially chosen from the compounds having the following formulae:

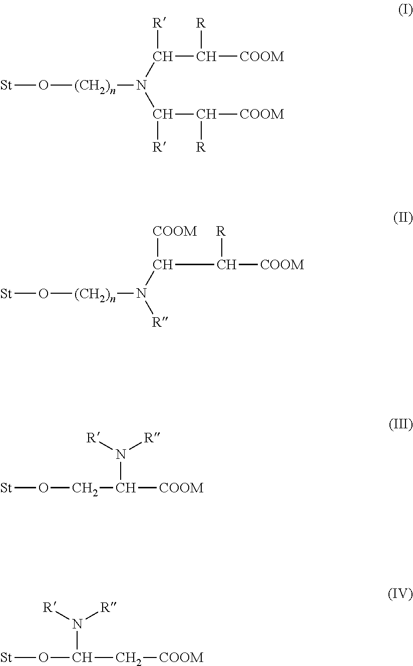

in which:
St-O represents a starch molecule;
R, which may be identical or different, represents a hydrogen atom or a methyl radical;
R′, which may be identical or different, represents a hydrogen atom, a methyl radical or a —COOH group;
n is an integer equal to 2 or 3;
M, which may be identical or different, denotes a hydrogen atom, an alkali metal or alkaline-earth metal such as Na, K, Li or NH₄, a quaternary ammonium or an organic amine;
R″ represents a hydrogen atom or an alkyl radical containing from 1 to 18 carbon atoms.

These compounds are especially described in patents U.S. Pat. No. 5,455,340 and US 4 017460.

The starch molecules may be derived from any plant source of starch, especially such as corn, potato, oat, rice, tapioca, sorghum, barley or wheat. It is also possible to use the hydrolysates of the starches mentioned above.

The modified starches are represented, for example, by the products sold under the names C*Tex-Instant (pregelatinized adipate), C*StabiTex-Instant (pregelatinized phosphate), C*PolarTex-Instant (pregelatinized hydroxypropyl), C*Set (acid hydrolysis, oxidation), C*size (oxidation), C*BatterCrisp (oxidation), C*DrySet (dextrinization), C*Tex™ (acetylated distarch adipate), C*PolarTex™ (hydroxypropyl distarch phosphate), C* StabiTex™ (distarch phosphate, acetylated distarch phosphate) by the company Cargill, by distarch phosphates or compounds that are rich in distarch phosphate, such as the product sold under the references Prejel VA-70-T AGGL (gelatinized hydroxypropyl cassava distarch phosphate) or Prejel TKI (gelatinized cassava distarch phosphate) or Prejel 200 (gelatinized acetylated cassava distarch phosphate) by the company Avebe or Structure Zea from National Starch (gelatinized corn distarch phosphate).

As examples of oxidized starches, use will be made especially of those sold under the name C*size from the company Cargill.

The native or modified starches described above may advantageously be used in a proportion of from 0.1% to 8% by weight of solids and preferably about 1% by weight, relative to the total weight of the aqueous phase.

Particulate Starches

Particulate starches that may be mentioned in particular include:
starches grafted with an acrylic polymer (homopolymer or copolymer) and in particular with sodium polyacrylate, for instance those sold under the name Sanfresh ST-100MC by the company Sanyo Chemical Industries or Makimousse 25 or Makimousse 12 by the company Daito Kasei (INCI name: Sodium polyacrylate starch);
hydrolysed starches grafted with an acrylic polymer (homopolymer or copolymer) and especially acryloacrylamide/sodium acrylate copolymer, for instance those sold under the names Water Lock A-240, A-180, B-204, D-223, A-100, C-200 and D-223 by the company Grain Processing (INCI name: Starch/acrylamide/sodium acrylate copolymer);
polymers based on starch, gum and cellulose derivative, such as the product containing starch and sodium carboxymethylcellulose, for instance the product sold under the name Lysorb 220 by the company Lysac.

Mention may be made most particularly of (C₁-C₄) carboxyalkyl starches, “carboxyalkylstarch”. These compounds are obtained by grafting carboxyalkyl groups onto one or more alcohol functions of starch, especially by reaction of starch and of sodium monochloroacetate in alkaline medium.

The carboxyalkyl groups are generally attached via an ether function, more particularly to carbon 1. The degree of substitution with carboxyalkyl units of the (C₁-C₄) carboxyalkyl starch preferably ranges from 0.1 to 1 and more particularly from 0.15 to 0.5. The degree of substitution is defined according to the present invention as being the mean number of hydroxyl groups substituted with an ester or ether group per monosaccharide unit of the polysaccharide.

The carboxyalkyl starches are advantageously used in the form of salts and especially salts of alkali metals or alkaline-earth metals such as Na, K, Li, NH₄, or salts of a quaternary ammonium or of an organic amine such as monoethanolamine, diethanolamine or triethanolamine. The (C₁-C₄) carboxyalkyl starches are, in the context of the present invention, advantageously carboxymethyl starches. The carboxymethyl starches preferably comprise units having the following formula:

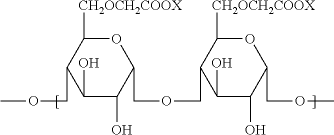

in which X, optionally covalently bonded to the carboxyl unit, denotes a hydrogen atom, an alkali metal or alkaline-earth metal such as Na, K, Li, NH₄, a quaternary ammonium or an organic amine, for instance monoethanolamine, diethanolamine or triethanolamine.

Preferably, X denotes an Na⁺ cation. The carboxyalkyl starches that may be used according to the present invention are preferably non-pregelatinized carboxyalkyl starches. The carboxyalkyl starches that may be used according to the present invention are preferably partially or totally crosslinked carboxyalkyl starches.

In general, a crosslinked carboxyalkyl starch has, as opposed to a non-crosslinked carboxyalkyl starch, an increased, controllable viscosity and greater stability. The crosslinking thus makes it possible to reduce the syneresis and to increase the resistance of the gel to shear effects.

The carboxyalkyl starches under consideration according to the invention are more particularly potato carboxyalkyl starches. Thus, the carboxyalkyl starches that may be used according to the present invention are preferably sodium salts of carboxyalkyl starches, in particular a sodium salt of potato carboxymethyl starch, sold especially under the name Primojel® by the company DMV International or Glycolys® and Glycolys® LV by the company Roquette.

According to one particular embodiment, the potato carboxymethyl starches sold especially under the name Glycolys® by the company Roquette will be used. As stated previously, the (C₁-C₄) carboxyalkyl starch particles are present in the compositions according to the invention in a swollen and unsplit form. This swelling may be characterized by a swelling power Q that may advantageously be between 10 and 30 ml/g and preferably between 15 and 25 ml (volume of liquid absorbed)/g of dry particulate material.

Thus, the size of the swollen carboxyalkyl starch particles used according to the present invention generally ranges from 25 to 300 μm. For example, the gel Primojel® containing 10% by weight of potato carboxyalkyl starch and sodium salt in water contains more than 80% of swollen particles of this starch with a diameter of greater than 50 microns and more particularly greater than 100 microns.

According to a preferred embodiment variant of the invention, these particles are used for the preparation of the compositions according to the invention, in this swollen particulate state. To do this, these particles are advantageously used in the form of an aqueous gel that is either prepared beforehand or already commercially available. The gels under consideration according to the invention are advantageously translucent.

For example, a carboxymethyl starch gel such as Primojelo which is at a concentration of 10% by weight may be adjusted to the required concentration before being used to prepare the expected cosmetic composition.

Such a particulate starch may be used in a proportion of from 0.1% to 5% by weight of solids relative to the total weight of the aqueous phase, preferably between 0.5% and 2.5% by weight and in particular in a proportion of about 1.5% by weight, relative to the total weight of the aqueous phase.

### Non-Starchy Polysaccharides

In general, the non-starchy polysaccharides may be chosen from polysaccharides produced by microorganisms; polysaccharides isolated from algae, higher plant polysaccharides, such as homogeneous polysaccharides, in particular celluloses and derivatives thereof or fructosans, heterogeneous polysaccharides such as gum arabics, galactomannans, glucomannans and pectins, and derivatives thereof; and mixtures thereof.

In particular, the polysaccharides may be chosen from fructans, gellans, glucans, amylose, amylopectin, glycogen, pullulan, dextrans, celluloses and derivatives thereof, in particular methylcelluloses, hydroxyalkylcelluloses, ethylhydroxyethylcelluloses and carboxymethylcelluloses, mannans, xylans, lignins, arabans, galactans, galacturonans, alginate-based compounds, chitin, chitosans, glucuronoxylans, arabinoxylans, xyloglucans, glucomannans, pectic acids and pectins, arabinogalactans, carrageenans, agars, glycosaminoglycans, gum arabics, tragacanth gums, ghatti gums, karaya gums, locust bean gums, galactomannans such as guar gums and nonionic derivatives thereof, in particular hydroxypropyl guar, and ionic derivatives thereof, biopolysaccharide gums of microbial origin, in particular scleroglucan or xanthan gums, mucopolysaccharides, and in particular chondroitin sulfates, and mixtures thereof.

These polysaccharides may be chemically modified, especially with urea or urethane groups, or by a hydrolysis, oxidation, esterification, etherification, sulfatation, phosphatation, amination, amidation or alkylation reaction, or by several of these modifications.

The derivatives obtained may be anionic, cationic, amphoteric or nonionic.

Advantageously, the polysaccharides may be chosen from carrageenans, in particular kappa-carrageenan, gellan gum, agar-agar, xanthan gum, alginate-based compounds, in particular sodium alginate, scleroglucan gum, guar gum, inulin and pullulan, and mixtures thereof.

In general, the compounds of this type that may be used in the present invention are chosen from those described especially in Kirk-Othmer's Encyclopedia of Chemical Technology, Third Edition, 1982, volume 3, pp. 896-900, and volume 15, pp. 439-458, in Polymers in Nature by E. A. McGregor and C. T. Greenwood, published by John Wiley & Sons, Chapter 6, pp. 240-328, 1980, in the publication by Robert L. Davidson entitled Handbook of Water-soluble Gums and Resins published by McGraw-Hill Book Company (1980) and in Industrial Gums—Polysaccharides and their Derivatives, edited by Roy L. Whistler, Second Edition, published by Academic Press Inc.

Such a gelling agent may be used in a proportion of from 0.1% to 8% by weight of solids relative to the total weight of the aqueous phase, especially from 0.1% to 6% by weight, preferably between 0.5% and 2.5% by weight, in particular in a proportion of about 1% or even in a proportion of about 1.5% by weight, relative to the total weight of the aqueous phase.

More precisely, these polysaccharides that are suitable for use in the invention may be distinguished according to whether they are derived from microorganisms, algae or higher plants, and are detailed below.

Polysaccharides Produced by Microorganisms

### Xanthan

Xanthan is a heteropolysaccharide produced on an industrial scale by the aerobic fermentation of the bacterium Xanthomonas campestris. Its structure is composed of a main chain of β-D-glucoses connected in β(1,4) manner, similar to cellulose. One glucose molecule out of two bears a trisaccharide side chain composed of an α-D-mannose, of a β-D-glucuronic acid and of a terminal β-D-mannose. The internal mannose residue is generally acetylated on carbon 6. Approximately 30% of the terminal mannose residues bear a pyruvate group linked in chelated form between carbons 4 and 6. The glucuronic acids and the charged pyruvic acids are ionizable and thus responsible for the anionic nature of xanthan (negative charge down to pH 1). The content of the pyruvate and acetate residues varies according to the bacterial strain, the fermentation process, the post-fermentation conditions and the purification stages. These groups can be neutralized in the commercial products with Na⁺, K⁺ or Ca²⁺ ions (Satia, 1986). The neutralized form can be converted into the acid form by ion exchange or by dialysis with an acid solution.

Xanthan gums have a molecular weight of between 1 000 000 and 50 000 000 and a viscosity of between 0.6 and 1.65 Pa·s for an aqueous composition comprising 1% of xanthan gum (measured at 25° C. using a Brookfield viscometer, LVT type, at 60 revolutions per minute).

Xanthan gums are represented, for example, by the products sold under the name Rhodicare by the company Rhodia Chimie, under the name Satiaxane™ by the company Cargill Texturizing Solutions (for the food, cosmetic and pharmaceutical industry), under the name Novaxan™ by the company ADM and under the names Kelzan® and Keltrola by the company CP-Kelco.

### Pullulan

Pullulan is a polysaccharide consisting of maltotriose units, known under the name α(1,4)-α(1,6)-glucan. Three glucose units in maltotriose are connected via an α(1,4) glycosidic bond, whereas the consecutive maltotriose units are connected to each other via an α(1,6) glycosidic bond.

Pullulan is produced, for example, under the reference Pullulan PF 20 by the company Hayashibara in Japan.

### Dextran and Dextran Sulfate

Dextran is a neutral polysaccharide not bearing any charge groups, which is biologically inert, prepared by fermentation of beet sugar containing only hydroxyl groups.

It is possible to obtain, from native dextran by hydrolysis and purification, dextran fractions of different molecular weights. Dextran may in particular be in the form of dextran sulfate.

Dextran is represented, for example, by the products sold under the name Dextran or Dextran T by the company Pharmacosmos, under the name Dextran 40 Powder or Dextran 70 Powder by the company Meito Sangyo Co. Dextran sulfate is sold by the company PK Chemical A/S under the name Dextran Sulfate.

### Succinoglycan

Succinoglycan is an extracellular polymer produced by bacterial fermentation, of high molecular weight, consisting of octasaccharide repeating units (8 repeating sugars). Succinoglycans are sold, for example, under the name Rheozan by the company Rhodia.

### Scleroglucan

Scleroglucan is a nonionic branched homopolysaccharide consisting of β-D glucan units. The molecules consist of a main linear chain formed by D-glucose units linked via β(1,3) bonds, and of which one in three units is linked to a D-glucose side unit via a β(1,6) bond.

A fuller description of scleroglucans and of their preparation may be found in document U.S. Pat. No. 3,301,848.

Scleroglucan is sold, for example, under the name Amigel by the company Alban Muller, or under the name Actigum™ CS by the company Cargill.

### Gellan Gum

Gellan gum is an anionic linear heteropolysaccharide based on oligosaccharide units composed of 4 saccharides (tetrasaccharide). D-glucose, L-rhamnose and D-glucuronic acid in 2/1/1 proportions are present in gellan gum in the form of monomer elements.

It is sold, for example, under the name Kelcogel CG LA by the company CP Kelco.

Polysaccharides Isolated from Algae

### Galactans

The polysaccharide according to the invention may be a galactan chosen especially from agar and carrageenans.

Carrageenans are anionic polysaccharides constituting the cell walls of various red algae (Rhodophyceae) belonging to the Gigartinaceae, Hypneaceae, Furcellariaceae and Polyideaceae families. They are generally obtained by hot aqueous extraction from natural strains of the said algae. These linear polymers, formed by disaccharide units, are composed of two D-galactopyranose units alternately linked via α(1,3) and β(1,4) bonds. These are highly sulfated polysaccharides (20-50%) and the α-D-galactopyranosyl residues may be in 3,6-anhydro form. According to the number and position of the ester sulfate groups on the repeat disaccharide of the molecule, several types of carrageenan are distinguished, namely: kappa-carrageenans, which bear one ester sulfate group, iota-carrageenans which bear two ester sulfate groups, and lambda-carrageenans which bear three ester sulfate groups.

Carrageenans are composed essentially of potassium, sodium, magnesium, triethanolamine and/or calcium salts and of ester sulfates of polysaccharides.

Carrageenans are sold especially by the company SEPPIC under the name Solagum®, by the company Gelymar under the names Carragel®, Carralact® and Carrasol®, by the company Cargill under the names Satiagel™ and Satiagum™, and by the company CP-Kelco under the names Genulacta®, Genugel® and Genuvisco®.

Galactans of agar type are galactose polysaccharides contained in the cell wall of some of these species of red algae (rhodophyceae). They are formed from a polymer group in which the base backbone is a β(1,3) D-galactopyranose and α(1,4) L 3-6 anhydrogalactose chain, these units repeating regularly and alternately. The differences within the agar family are due to the presence or absence of methyl or carboxyethyl solvated groups. These hybrid structures are generally present in variable percentage, depending on the species of algae and the season of harvest.

Agar-agar is a mixture of polysaccharides (agarose and agaropectin) of high molecular mass, between 40 000 and 300 000 g·mol⁻¹. It is obtained by manufacturing algal extraction juices, generally by autoclaving, and by treating these juices comprising about 2% agar-agar, in order to extract the latter.

Agar is produced, for example, by the group B&V Agar Producers under the names Gold Agar, Agarite and Grand Agar by the company Hispanagar, and under the names Agar-Agar, QSA (Quick Soluble Agar) and Puragar by the company Setexam.

### Furcellaran

Furcellaran is obtained commercially from red algae Furcellaria fasztigiata. Furcellaran is produced, for example, by the company Est-Agar.

### Alginate-Based Compound

For the purposes of the invention, the term “alginate-based compound” means alginic acid, alginic acid derivatives and the salts of alginic acid (alginates) or of the said derivatives.

Preferably, the alginate-based compound is water-soluble.

Alginic acid, a natural substance derived from brown algae or certain bacteria, is a polyuronic acid composed of two uronic acids linked by 1,4-glycosidic bonds: β-D-mannuronic (M) acid and α-L-glucuronic (G) acid.

Alginic acid is capable of forming water-soluble salts (alginates) with alkali metals such as sodium, potassium or lithium, substituted cations of lower amines and of ammonium such as methylamine, ethanolamine, diethanolamine or triethanolamine. These alginates are water-soluble in aqueous medium at a pH equal to 4 but dissociate into alginic acid at a pH below 4.

This (these) alginate-based compound(s) are capable of crosslinking in the presence of at least one crosslinking agent, by formation of ionic bonds between the said alginate-based compound(s) and the said crosslinking agent(s). The formation of multiple crosslinking between several molecules of the said alginate-based compound(s) leads to the formation of a water-insoluble gel.

Use is preferably made of alginate-based compounds with a weight-average molecular mass ranging from 10 000 to 1 000 000, preferably from 15 000 to 500 000 and better still from 20 000 to 250 000.

According to a preferred embodiment, the alginate-based compound is alginic acid and/or a salt thereof.

Advantageously, the alginate-based compound is an alginate salt, and preferably sodium alginate.

The alginate-based compound may be chemically modified, especially with urea or urethane groups, or by a hydrolysis, oxidation, esterification, etherification, sulfatation, phosphatation, amination, amidation or alkylation reaction, or by several of these modifications.

The derivatives obtained may be anionic, cationic, amphoteric or nonionic.

The alginate-based compounds that are suitable for use in the invention may be represented, for example, by the products sold under the names Kelcosol, Satialgine™, Cecalgum™ or Algogel™ by the company Cargill Products, under the name Protanal™ by the company FMC Biopolymer, under the name Grindsted® Alginate by the company Danisco, under the name Kimica Algin by the company Kimica, and under the names Manucol® and Manugel® by the company ISP.

Polysaccharides from Higher Plants

This category of polysaccharides may be divided into homogeneous polysaccharides (only one species of saccharide) and heterogeneous polysaccharides composed of several types of saccharide.

a) Homogeneous Polysaccharides and Derivatives Thereof

The polysaccharide according to the invention may be chosen from celluloses and derivatives or fructosans.

### Celluloses and Derivatives

The polysaccharide according to the invention may also be a cellulose or a derivative thereof, especially cellulose ethers or esters (e.g.: methylcellulose, carboxymethylcellulose, hydroxymethylcellulose, hydroxyethylcellulose, hydroxypropylcellulose, hydroxymethylpropylcellulose, cellulose acetate, cellulose nitrate, nitrocellulose).

The invention may also contain a cellulose-based associative polymer. According to the invention, the term “cellulose-based compound” means any polysaccharide compound bearing in its structure linear sequences of anhydroglucopyranose (AGU) residues linked via β(1,4) glycosidic bonds. The repeating unit is the cellobiose dimer. AGUs are found in chair conformation and bear three hydroxyl functions: two secondary alcohols (in positions 2 and 3) and a primary alcohol (in position 6). The polymers thus formed combine together via intermolecular bonds of hydrogen bonding type, thus giving the cellulose a fibrillar structure (about 1500 molecules per fibre).

The degree of polymerization differs enormously according to the origin of the cellulose; its value may range from a few hundred to a few tens of thousands.

Cellulose has the following chemical structure:

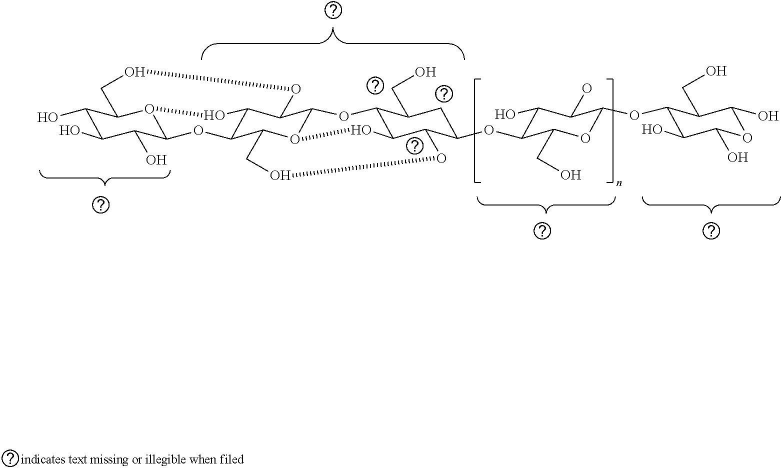

The hydroxyl groups of cellulose may react partially or totally with different chemical reagents to give cellulose derivatives having intrinsic properties. The cellulose derivatives may be anionic, cationic, amphoteric or nonionic. Among these derivatives, cellulose ethers, cellulose esters and cellulose ester ethers are distinguished.

Among the nonionic cellulose ethers, mention may be made of alkylcelluloses such as methylcelluloses and ethylcelluloses, hydroxyalkylcelluloses such as hydroxymethylcelluloses, hydroxyethylcelluloses and hydroxypropylcelluloses, and mixed hydroxyalkylalkylcelluloses such as hydroxypropylmethylcelluloses, hydroxyethylmethylcelluloses, hydroxyethylethylcelluloses and hydroxybutylmethylcelluloses.

Among the anionic cellulose ethers, mention may be made of carboxyalkylcelluloses and salts thereof. By way of example, mention may be made of carboxymethylcelluloses, carboxymethylmethylcelluloses and carboxymethylhydroxyethylcelluloses and sodium salts thereof.

Among the cationic cellulose ethers, mention may be made of crosslinked or non-crosslinked, quaternized hydroxyethylcelluloses.

The quaternizing agent may in particular be glycidyltrimethylammonium chloride or a fatty amine such as laurylamine or stearylamine. Another cationic cellulose ether that may be mentioned is hydroxyethylcellulosehydroxypropyltrimethylammonium.

The quaternized cellulose derivatives are, in particular:
quaternized celluloses modified with groups comprising at least one fatty chain, such as alkyl, arylalkyl or alkylaryl groups comprising at least 8 carbon atoms, or mixtures thereof;
quaternized hydroxyethylcelluloses modified with groups comprising at least one fatty chain, such as alkyl, arylalkyl or alkylaryl groups comprising at least 8 carbon atoms, or mixtures thereof.

The alkyl radicals borne by the above quaternized celluloses or hydroxyethylcelluloses preferably comprise from 8 to 30 carbon atoms. The aryl radicals preferably denote phenyl, benzyl, naphthyl or anthryl groups.

Examples of quaternized alkylhydroxyethylcelluloses containing C8-C30 fatty chains that may be indicated include the products Quatrisoft LM 200, Quatrisoft LM-X 529-18-A, Quatrisoft LM-X 529-18B (C12 alkyl) and Quatrisoft LM-X 529-8 (C₁₈ alkyl) sold by the company Amerchol, and the products Crodacel Q M, Crodacel Q L (C₁₂ alkyl) and Crodacel QS (C₁₈ alkyl) sold by the company Croda.

Among the cellulose derivatives, mention may also be made of:
celluloses modified with groups comprising at least one fatty chain, for instance hydroxyethylcelluloses modified with groups comprising at least one fatty chain, such as alkyl groups, especially of C₈-C₂₂, arylalkyl and alkylaryl groups, such as Natrosol Plus Grade 330 CS (C₁₆ alkyls) sold by the company Aqualon, and
celluloses modified with alkylphenyl polyalkylene glycol ether groups, such as the product Amercell Polymer HM-1500 (nonylphenyl polyethylene glycol (15) ether) sold by the company Amerchol.

Among the cellulose esters are mineral esters of cellulose (cellulose nitrates, sulfates, phosphates, etc.), organic esters of cellulose (cellulose monoacetates, triacetates, amidopropionates, acetatebutyrates, acetatepropionates or acetatetrimellitates, etc.), and mixed organic/mineral esters of cellulose, such as cellulose acetatebutyrate sulfates and cellulose acetatepropionate sulfates. Among the cellulose ester ethers, mention may be made of hydroxypropylmethylcellulosc phthalates and ethylcellulose sulfates.

The cellulose-based compounds of the invention may be chosen from unsubstituted celluloses and substituted celluloses.

The celluloses and derivatives are represented, for example, by the products sold under the names Avicel® (microcrystalline cellulose, MCC) by the company FMC Biopolymers, under the name Cekol (carboxymethylcellulose) by the company Noviant (CP-Kelco), under the name Akucell AF (sodium carboxymethylcellulose) by the company Akzo Nobel, under the name Methocel™ (cellulose ethers) and Ethocel™ (ethylcellulose) by the company Dow, and under the names Aqualon® (carboxymethylcellulose and sodium carboxymethylcellulose), Benecele® (methylcellulose), Blanose™ (carboxymethylcellulose), Culminal® (methylcellulose, hydroxypropylmethylcellulose), Klucel® (hydroxypropylcellulose), Polysurf® (cetylhydroxyethylcellulose) and Natrosol® CS (hydroxyethylcellulose) by the company Hercules Aqualon.

### Fructosans

The polysaccharide according to the invention may especially be a fructosan chosen from inulin and derivatives thereof (especially dicarboxy and carboxymethyl inulins).

Fructans or fructosans are oligosaccharides or polysaccharides comprising a sequence of anhydrofructose units optionally combined with several saccharide residues other than fructose. Fructans may be linear or branched. Fructans may be products obtained directly from a vegetable or microbial source or alternatively products whose chain length has been modified (increased or decreased) by fractionation, synthesis or hydrolysis, in particular enzymatic. Fructans generally have a degree of polymerization from 2 to about 1000 and preferably from 2 to about 60.

Three groups of fructans are distinguished. The first group corresponds to products whose fructose units are for the most part linked via β(2,1) bonds. These are essentially linear fructans such as inulins.

The second group also corresponds to linear fructoses, but the fructose units are essentially linked via β(2,6) bonds. These products are levans.

The third group corresponds to mixed fructans, i.e. fructans containing β(2,6) and β(2,1) sequences. These are essentially branched fructans, such as graminans.

The fructans preferred in the compositions according to the invention are inulins. Inulin may be obtained, for example, from chicory, dahlia or Jerusalem artichoke, preferably from chicory.

In particular, the polysaccharide, especially the inulin, has a degree of polymerization from 2 to about 1000 and preferably from 2 to about 60, and a degree of substitution of less than 2 on the basis of one fructose unit.

The inulin used for this invention is represented, for example, by the products sold under the name Beneo™ Inulin by the company Orafti, and under the name Frutafit® by the company Sensus.
b) Heterogeneous Polysaccarides and Derivatives Thereof

Polysaccharides that may be used according to the invention may be gums, for instance cassia gum, karaya gum, konjac gum, gum tragacanth, tara gum, acacia gum or gum arabic.

### Gum Arabic

Gum arabic is a highly branched acidic polysaccharide which is present in the form of mixtures of potassium, magnesium and calcium salts. The monomer elements of the free acid (arabic acid) are D-galactose, L-arabinose, L-rhamnose and D-glucuronic acid.

Galactomannans (guar, locust bean, fenugreek, tara gum) and derivatives (phosphated guar, hydroxypropyl guar, etc.)

Galactomannans are nonionic polysaccharides extracted from the albumin of seeds of leguminous plants, of which they constitute the storage carbohydrate.

Galactomannans are macromolecules consisting of a main chain of D-mannopyranose units connected in β(1,4) fashion, carrying side branches consisting of a single D-galactopyranose unit connected in α(1,6) fashion to the main chain. The various galactomannans differ, on the one hand, in the proportion of α-D-galactopyranose units present in the polymer and, on the other hand, in significant differences in terms of distribution of the galactose units along the mannose chain.

The mannose/galactose (M/G) ratio is of the order of 2 for guar gum, of 3 for tara gum and of 4 for locust bean gum.

Galactomannans have the following chemical structure:

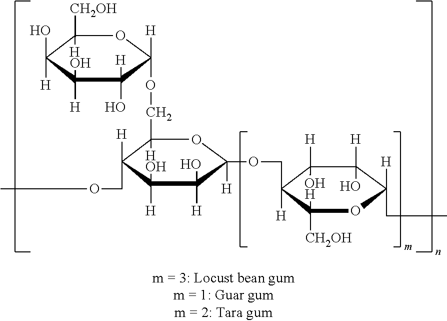

### Guar

Guar gum is characterized by a mannose:galactose ratio of the order of 2:1. The galactose group is uniformly distributed along the mannose chain.

The guar gums that may be used according to the invention may be nonionic, cationic or anionic. According to the invention, use may be made of unmodified or chemically modified nonionic guar gums.

Unmodified nonionic guar gums are, for example, the products sold under the names Vidogum GH, Vidogum G and Vidocrem by the company Unipektin and under the name Jaguar by the company Rhodia, under the name Meypro® Guar by the company Danisco, under the name Viscogum™ by the company Cargill and under the name Supercol® Guar Gum by the company Aqualon.

The hydrolysed nonionic guar gums that may be used according to the invention are represented, for example, by the products sold under the name Meyprodor® by the company Danisco.

The modified nonionic guar gums that may be used according to the invention are preferably modified with C₁-C₆ hydroxyalkyl groups, among which mention may be made, for example, of hydroxymethyl, hydroxyethyl, hydroxypropyl and hydroxybutyl groups.

Such nonionic guar gums optionally modified by hydroxyalkyl groups are sold, for example, under the trade names Jaguar HP 60, Jaguar HP 105 and Jaguar HP 120 (hydroxypropyl guar) by the company Rhodia or under the name N-Hance® HP (hydroxypropyl guar) by the company Aqualon.

The cationic galactomannan gums preferably have a cationic charge density of less than or equal to 1.5 meq/g and more particularly of between 0.1 and 1 meq/g. The charge density can be determined according to the Kjeldahl method. It generally corresponds to a pH of the order of 3 to 9.

Generally, for the purposes of the present invention, the term “cationic galactomannan gum” means any galactomannan gum containing cationic groups and/or groups that can be ionized into cationic groups.

The preferred cationic groups are chosen from those comprising primary, secondary, tertiary and/or quaternary amine groups.

The cationic galactomannan gums used generally have a weight-average molecular mass of between 500 and 5×10⁶ approximately and preferably of between 10³ and 3×10⁶ approximately.

The cationic galactomannan gums that may be used according to the present invention are, for example, gums comprising tri(C₁-C₄)alkylammonium cationic groups. Preferably, 2% to 30% by number of the hydroxyl functions of these gums bear trialkylammonium cationic groups.

Mention may very particularly be made, among these trialkylammonium groups, of the trimethylammonium and triethylammonium groups.

Even more preferentially, these groups represent from 5% to 20% by weight relative to the total weight of the modified galactomannan gum.

According to the invention, the cationic galactomannan gum is preferably a guar gum comprising hydroxypropyltrimethylammonium groups, i.e. a guar gum modified, for example, with 2,3-epoxypropyltrimethylammonium chloride.

These galactomannan gums, in particular guar gums modified by cationic groups, are products already known per se and are, for example, described in the patents U.S. Pat. Nos. 3,589,578 and 4,031,307. Such products are furthermore sold in particular under the trade names of Jaguar Excel, Jaguar C13 S, Jaguar C 15, Jaguar C 17 and Jaguar C162 (Guar Hydroxypropyltrimonium Chloride) by the company Rhodia, under the name Amilan® Guar (Guar Hydroxypropyltrimonium Chloride) by the company Degussa and under the name N-Hance® 3000 (Guar Hydroxypropyltrimonium Chloride) by the company Aqualon.

The anionic guar gums that may be used according to the invention are polymers comprising groups derived from carboxylic acid, sulfonic acid, sulfenic acid, phosphoric acid, phosphonic acid or pyruvic acid. Preferably, the anionic group is a carboxylic acid group. The anionic group may also be in the form of an acid salt, especially a sodium, calcium, lithium or potassium salt.

The anionic guar gums that may be used according to the invention are preferentially carboxymethyl guar derivatives (carboxymethyl guar or carboxymethyl hydroxypropyl guar).

### Locust Bean

Locust bean gum is extracted from the seeds of the carob tree (Ceratonia siliqua).

The unmodified locust bean gum that may be used in this invention is sold, for example, under the name Viscogum™ by the company Cargill, under the name Vidogum L by the company Unipektin or under the name Grinsted LBG by the company Danisco.

The chemically modified locust bean gums that may be used in this invention may be represented, for example, by the cationic locust beans sold under the name Catinal CLB (Locust Bean Hydroxypropyltrimonium Chloride) by the company Toho.

### Tara Gum

The tara gum that may be used in the context of this invention is sold, for example, under the name Vidogum SP by the company Unipektin.

Glucomannans (konjac Gum)

Glucomannan is a polysaccharide of high molecular weight (500 000<Mglucomannan <2 000 000), composed of D-mannose and D-glucose units with a branch approximately every 50 or 60 units. It is found in wood but it is also the main constituent of konjac gum. Konjac (Amorphophallus konjac) is a plant of the Araceae family.

The products that may be used according to the invention are sold, for example, under the names Propol® and Rheolex® by the company Shimizu.

### LM and HM Pectins And Derivatives

Pectins are linear polymers of α-D-galacturonic acid (at least 65%) linked in positions 1 and 4, with a certain proportion of carboxylic groups esterified with a methanol group. About 20% of the sugars constituting the pectin molecule are neutral sugars (L-rhamnose, D-glucose, D-galactose, L-arabinose, D-xylose). The L-rhamnose residues are present in all pectins, integrated into the main chain in positions 1,2.

The uronic acid molecules bear carboxyl functions. This function gives the pectins the capacity for exchanging ions, when they are in COO⁻ form. Bivalent ions (in particular calcium) have the capacity of forming ionic bridges between two carboxyl groups of two different pectin molecules.

In the natural state, a certain proportion of the carboxylic groups are esterified with a methanol group. The natural degree of esterification of a pectin may range between 70% (apple, lemon) and 10% (strawberry) according to the source used. Starting with pectins with a high degree of esterification, it is possible to hydrolyse the —COOCH₃ group, so as to obtain weakly esterified pectins. Depending on the proportion of methylated or non-methylated monomers, the chain is therefore more or less acidic. Pectins are thus defined as being HM (high-methoxy) pectins, having a degree of esterification of greater than 50%, and LM (low-methoxy) pectins, having a degree of esterification of less than 50%.

In the case of amide pectins, the —OCH₃ group is substituted with a —NH₂ group.

Pectins are especially sold by the company Cargill under the name Unipectine™, by the company CP-Kelco under the name Genu, and by Danisco under the name Grinsted Pectin.

### Other Polysaccharides

Among the other polysaccharides that may be used according to the invention, mention may also be made of chitin (poly-N-acetyl-D-glucosamine, β(1,4)-2-acetamido-2-deoxy-D-glucose), chitosan and derivatives (chitosan β-glycerophosphate, carboxymethylchitin, etc.) such as those sold by the company France-Chitine; glycosaminoglycans (GAG) such as hyaluronic acid, chondroitin sulfate, dermatan sulfate and keratan sulfate, and preferably hyaluronic acid; xylans (or arabinoxylans) and derivatives.

Arabinoxylans are polymers of xylose and arabinose, which are grouped together under the name “penlosans”.

Xylans consist of a main chain of D-xylose units linked in β(1,4) manner, and on which are found three substituents (Rouau & Thibault, 1987): acid units, α-L-arabinofuranose units, side chains which may contain arabinose, xylose, galactose and glucuronic acid.

According to this variant, the polysaccharide is preferably hyaluronic acid, or a salt thereof such as the sodium salt (sodium hyaluronate).

### II. Synthetic Polymeric Gelling Agents

A hydrophilic gelling agent may be at least one synthetic polymeric gelling agent chosen from crosslinked acrylic homopolymers or copolymers; associative polymers, in particular associative polymers of polyurethane type; polyacrylamides and crosslinked and/or neutralized 2-acrylamido-2-methylpropanesulfonic acid polymers and copolymers; modified or unmodified carboxyvinyl polymers, and mixtures thereof.

For the purposes of the invention, the term “synthetic” means that the polymer is neither naturally existing nor a derivative of a polymer of natural origin.

The synthetic polymeric hydrophilic gelling agent under consideration according to the invention may or may not be particulate.

For the purposes of the invention, the term “particulate” means that the polymer is in the form of particles, preferably spherical particles.

II.A Particulate Synthetic Polymeric Gelling Agents

They are preferably chosen from crosslinked polymers.

They may especially be crosslinked acrylic homopolymers or copolymers, which are preferably partially neutralized or neutralized, and which are in particulate form.

According to one embodiment, the particulate gelling agent according to the present invention is chosen from crosslinked sodium polyacrylates. Preferably, it has in the dry or non-hydrated state a mean size of less than or equal to 100 μm and preferably less than or equal to 50 μm. The mean size of the particles corresponds to the mass-average diameter (D50) measured by laser particle size analysis or another equivalent method known to those skilled in the art.

Thus, preferably, the particulate gelling agent according to the present invention is chosen from crosslinked sodium polyacrylates, preferably in the form of particles with a mean size (or mean diameter) of less than or equal to 100 microns, more preferably in the form of spherical particles.

As examples of crosslinked sodium polyacrylates, mention may be made of those sold under the brand names Octacare X100, X110 and RM100 by the company Avecia, those sold under the names Flocare GB300 and Flosorb 500 by the company SNF, those sold under the names Luquasorb 1003, Luquasorb 1010, Luquasorb 1280 and Luquasorb 1110 by the company BASF, those sold under the names Water Lock G400 and G430 (INCI name: Acrylamide/Sodium acrylate copolymer) by the company Grain Processing.

Mention may also be made of crosslinked polyacrylate microspheres, for instance those sold under the name Aquakeep® 10 SH NF by the company Sumitomo Seika.

Such gelling agents may be used in a proportion of from 0.1% to 5% by weight of solids relative to the total weight of the aqueous phase, especially from 0.5% to 2% by weight and in particular in a proportion of about from 0.8% to 1.7% by weight, relative to the total weight of the aqueous phase.

II.B Non-Particulate Synthetic Polymeric Gelling Agents

This family of gelling agents may be detailed under the following subfamilies:

1. associative polymers,

2. polyacrylamides and crosslinked and/or neutralized 2-acrylamido-2-methylpropanesulfonic acid polymers and copolymers, and

3. modified or unmodified carboxyvinyl polymers.

1. Associative Polymers

For the purposes of the present invention, the term “associative polymer” means any amphiphilic polymer comprising in its structure at least one fatty chain and at least one hydrophilic portion. The associative polymers in accordance with the present invention may be anionic, cationic, nonionic or amphoteric.

### Associative Anionic Polymers

Among the associative anionic polymers that may be mentioned are those comprising at least one hydrophilic unit, and at least one fatty-chain allyl ether unit, more particularly those whose hydrophilic unit is formed by an unsaturated ethylenic anionic monomer, more particularly by a vinylcarboxylic acid and most particularly by an acrylic acid or a methacrylic acid or mixtures thereof, and whose fatty-chain allyl ether unit corresponds to the monomer of formula (I) below:

CH₂═C(R′)CH₂O BₙR (I)

in which R′ denotes H or CH₃, B denotes the ethylenoxy radical, n is zero or denotes an integer ranging from 1 to 100, R denotes a hydrocarbon-based radical chosen from alkyl, arylalkyl, aryl, alkylaryl and cycloalkyl radicals, comprising from 8 to 30 carbon atoms, preferably 10 to 24 and even more particularly from 12 to 18 carbon atoms.

Anionic amphiphilic polymers of this type are described and prepared, according to an emulsion polymerization process, in patent EP 0 216 479.

Among the associative anionic polymers that may also be mentioned are maleic anhydride/C₃₀-C₃₈ α-olefin/alkyl maleate terpolymers, such as the product (maleic anhydride/C₃₀-C₃₈ (α-olefin/isopropyl maleate copolymer) sold under the name Performa V 1608 by the company Newphase Technologies.

Among the associative anionic polymers, it is possible, according to a preferred embodiment, to use copolymers comprising among their monomers an α,β-monoethylenically unsaturated carboxylic acid and an ester of an α,β-monoethylenically unsaturated carboxylic acid and of an oxyalkylenated fatty alcohol.

Preferentially, these compounds also comprise as monomer an ester of an α,β-monoethylenically unsaturated carboxylic acid and of a C₁-C₄ alcohol.

Examples of compounds of this type that may be mentioned include Aculyn 22® sold by the company Röhm & Haas, which is a methacrylic acid/ethyl acrylate/oxyalkylenated stearyl methacrylate (comprising 20 OE units) terpolymer or Aculyn 28 (methacrylic acid/ethyl acrylate/oxyethylenated behenyl methacrylate (25 OE) terpolymer).

Examples of associative anionic polymers that may also be mentioned include anionic polymers comprising at least one hydrophilic unit of unsaturated olefinic carboxylic acid type, and at least one hydrophobic unit exclusively of the type such as a (C₁₀-C₃₀) alkyl ester of an unsaturated carboxylic acid. Examples that may be mentioned include the anionic polymers described and prepared according to patents U.S. Pat. No. 3,915,921 and 4 509 949.

Associative anionic polymers that may also be mentioned include anionic terpolymers.

The anionic terpolymer used according to the invention is a linear or branched and/or crosslinked terpolymer, of at least one monomer (1) bearing an acid function in free form, which is partially or totally salified with a nonionic monomer (2) chosen from N,N-dimethylacrylamide and 2-hydroxyethyl acrylate and at least one polyoxyethylenated alkyl acrylate monomer (3) of formula (I) below:

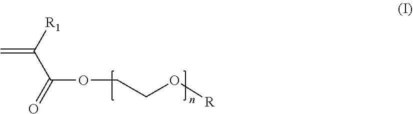

in which R1 represents a hydrogen atom, R represents a linear or branched C₂-C₈ alkyl radical and n represents a number ranging from 1 to 10.

The term “branched polymer” denotes a non-linear polymer which bears side chains so as to obtain, when this polymer is dissolved in water, a high degree of entanglement leading to very high viscosities, at a low speed gradient.

The term “crosslinked polymer” denotes a non-linear polymer which is in the form of a three-dimensional network that is insoluble in water but swellable in water, leading to the production of a chemical gel.

The acid function of the monomer (1) is especially a sulfonic acid or phosphonic acid function, the said functions being in free or partially or totally salified form.

The monomer (1) may be chosen from styrenesulfonic acid, ethylsulfonic acid and 2-methyl-2-[(1-oxo-2-propenyl)amino]-1-propanesulfonic acid (also known as acryloyldimethyl taurate), in free or partially or totally salified form. It is present in the anionic terpolymer preferably in molar proportions of between 5 mol % and 95 mol % and more particularly between 10 mol % and 90 mol %. The monomer (1) will more particularly be 2-methyl-2-[(l-oxo-2-propenyl)amino]-1-propanesulfonic acid in free or partially or totally salified form.

The acid function in partially or totally salified form will preferably be an alkali metal salt such as a sodium or potassium salt, an ammonium salt, an amino alcohol salt such as a monoethanolamine salt, or an amino acid salt such as a lysine salt.

The monomer (2) is preferably present in the anionic terpolymer in molar proportions of between 4.9 mol % and 90 mol %, more particularly between 9.5 mol % and 85 mol % and even more particularly between 19.5 mol % and 75 mol %.

In formula (I), examples of linear C₈-C₁₆ alkyl radicals that may be mentioned include octyl, decyl, undecyl, tridecyl, tetradecyl, pentadecyl and hexadecyl.

In formula (I), examples of branched C₈-C₁₆ alkyl radicals that may be mentioned include 2-ethylhexyl, 2-propylheptyl, 2-butyloctyl, 2-pentylnonyl, 2-hexyldecyl, 4-methylpentyl, 5-methylhexyl, 6-methylheptyl, 15-methylpentadecyl, 16-methylheptadecyl and 2-hexyloctyl.

According to a particular form of the invention, in formula (I), R denotes a C₁₂-C₁₆ alkyl radical.

According to a particular form of the invention, in formula (I), n ranges from 3 to 5.

Tetraethoxylated lauryl acrylate will more particularly be used as monomer of formula (I).

The monomer (3) of formula (I) is preferably present in the anionic terpolymer in molar proportions of between 0.1 mol % and 10 mol % and more particularly between 0.5 mol % and 5 mol %.

According to a particular mode of the invention, the anionic terpolymer is crosslinked and/or branched with a diethylenic or polyethylenic compound in the proportion expressed relative to the total amount of monomers used, from 0.005 mol % to 1 mol %, preferably from 0.01 mol % to 0.5 mol % and more particularly from 0.01 mol % to 0.25 mol %.

The crosslinking agent and/or branching agent is preferably chosen from ethylene glycol dimethacrylate, diallyloxyacetic acid or a salt thereof, such as sodium diallyloxyacetate, tetraallyloxyethane, ethylene glycol diacrylate, diallylurea, triallylamine, trimethylolpropane triacrylate and methylenebis(acrylamide), or mixtures thereof.

The anionic terpolymer may contain additives such as complexing agents, transfer agents or chain-limiting agents.

Use will be made more particularly of an anionic terpolymer of 2-methyl-2-[(1-oxo-2-propenyl]amino]-1-propanesulfonic acid partially or totally salified in the form of the ammonium salt, N,N-dimethylacrylamide and tetraethoxylated lauryl acrylate crosslinked with trimethylolpropane triacrylate, of INCI name Polyacrylate Crosspolymer-6, such as the product sold under the trade name Sepimax Zen® by the company SEPPIC.

### Cationic Associative Polymer

Cationic associative polymers that may be mentioned include polyacrylates bearing amine side groups.

The polyacrylates bearing quaternized or non-quaternized amine side groups contain, for example, hydrophobic groups of the type such as steareth-20 (polyoxyethylenated (20) stearyl alcohol).

Examples of polyacrylates bearing amino side chains that may be mentioned are the polymers 8781-121B or 9492-103 from the company National Starch.

### Nonionic Associative Polymers

The nonionic associative polymers may be chosen from:
copolymers of vinylpyrrolidone and of fatty-chain hydrophobic monomers;
copolymers of C₁-C₆ alkyl methacrylates or acrylates and of amphiphilic monomers comprising at least one fatty chain;
copolymers of hydrophilic methacrylates or acrylates and of hydrophobic monomers comprising at least one fatty chain, for instance the polyethylene glycol methacrylate/lauryl methacrylate copolymer;
associative polyurethanes.

Associative polyurethanes are nonionic block copolymers comprising in the chain both hydrophilic blocks usually of polyoxyethylene nature (polyurethanes may then be referred to as polyurethane polyethers), and hydrophobic blocks that may be aliphatic sequences alone and/or cycloaliphatic and/or aromatic sequences.

In particular, these polymers comprise at least two hydrocarbon-based lipophilic chains containing from 6 to 30 carbon atoms, separated by a hydrophilic block, the hydrocarbon-based chains possibly being pendent chains or chains at the end of the hydrophilic block. In particular, it is possible for one or more pendent chains to be envisioned. In addition, the polymer may comprise a hydrocarbon-based chain at one end or at both ends of a hydrophilic block.

Associative polyurethanes may be block polymers, in triblock or multiblock form. The hydrophobic blocks may thus be at each end of the chain (for example: triblock copolymer containing a hydrophilic central block) or distributed both at the ends and in the chain (for example: multiblock copolymer). These polymers may also be graft polymers or star polymers. Preferably, the associative polyurethanes are triblock copolymers in which the hydrophilic block is a polyoxyethylene chain comprising from 50 to 1000 oxyethylene groups. In general, associative polyurethanes comprise a urethane bond between the hydrophilic blocks, whence arises the name.

According to a preferred embodiment, a nonionic associative polymer of polyurethane type is used as gelling agent.

As examples of nonionic fatty-chain polyurethane polyethers that may be used in the invention, it is also possible to use Rheolate® FX 1100 (Steareth-100/PEG 136/HDI (hexamethyl diisocyanate) copolymer), Rheolate® 205 containing a urea function, sold by the company Elementis, or Rheolate® 208, 204 or 212, and also Acrysol® RM 184 or Acrysol® RM 2020.

Mention may also be made of the product Elfacos® T210 containing a C12-C₁₄ alkyl chain, and the product Elfacos® T212 containing a C₁₆₋₁₈ alkyl chain (PPG-14 Palmeth-60 Hexyl Dicarbamate), from Akzo.

The product DW 1206B® from Röhm & Haas containing a C₂₀ alkyl chain and a urethane bond, sold at a solids content of 20% in water, may also be used.

Use may also be made of solutions or dispersions of these polymers, especially in water or in aqueous-alcoholic medium. Examples of such polymers that may be mentioned are Rheolate® 255, Rheolate® 278 and Rheolate® 244 sold by the company Elementis. Use may also be made of the products DW 1206F and DW 1206J sold by the company Röhm & Haas.

The associative polyurethanes that may be used according to the invention are in particular those described in the article by G. Fonnum, J. Bakke and Fk. Hansen-Colloid Polym. Sci., 271, 380-389 (1993).

Even more particularly, according to the invention, use may also be made of an associative polyurethane that may be obtained by polycondensation of at least three compounds comprising (i) at least one polyethylene glycol comprising from 150 to 180 mol of ethylene oxide, (ii) stearyl alcohol or decyl alcohol, and (iii) at least one diisocyanate.

Such polyurethane polyethers are sold especially by the company Röhm & Haas under the names Aculyn® 46 and Aculyn® 44; Aculyn® 46 is a polycondensate of polyethylene glycol containing 150 or 180 mol of ethylene oxide, of stearyl alcohol and of methylenebis(4-cyclohexyl isocyanate) (SMDI), at 15% by weight in a matrix of maltodextrin (4%) and water (81%), and Aculyn® 44 is a polycondensate of polyethylene glycol containing 150 or 180 mol of ethylene oxide, of decyl alcohol and of methylenebis(4-cyclohexyl isocyanate) (SMDI), at 35% by weight in a mixture of propylene glycol (39%) and water (26%).

Use may also be made of solutions or dispersions of these polymers, especially in water or in aqueous-alcoholic medium. Examples of such polymers that may be mentioned include SER AD FX1010, SER AD FX1035 and SER AD 1070 from the company Elementis, and Rheolate® 255, Rheolate® 278 and Rheolate® 244 sold by the company Elementis. Use may also be made of the products Aculyn® 44, Aculyn® 46, DW 1206F and DW 1206J, and also Acrysol® RM 184 from the company Röhm & Haas, or alternatively Borchi Gel LW 44 from the company Borchers, and mixtures thereof.

### Amphoteric Associative Polymers

Among the associative amphoteric polymers of the invention, mention may be made of crosslinked or non-crosslinked, branched or unbranched amphoteric polymers, which may be obtained by copolymerization:

1) of at least one monomer of formula (IVa) or (IVb):

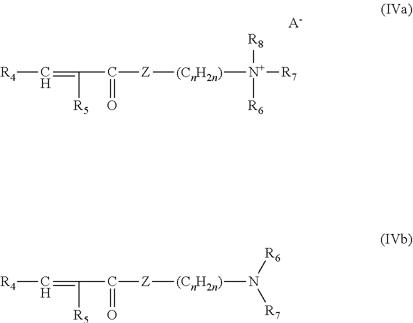

in which R4 and R5, which may be identical or different, represent a hydrogen atom or a methyl radical;

R₆, R₇ and R₈, which may be identical or different, represent a linear or branched alkyl radical containing from 1 to 30 carbon atoms;

Z represents an NH group or an oxygen atom;

n is an integer from 2 to 5;

A′ is an anion derived from an organic or mineral acid, such as a methosulfate anion or a halide such as chloride or bromide;

2) of at least one monomer of formula (V):

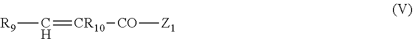

in which R9 and R10, which may be identical or different, represent a hydrogen atom or a methyl radical;

Z₁ represents a group OH or a group NHC(CH₃)₂CH₂SO₃H;

3) of at least one monomer of formula (VI):

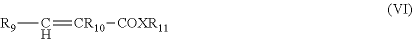

in which R9 and R10, which may be identical or different, represent a hydrogen atom or a methyl radical, X denotes an oxygen or nitrogen atom and R11 denotes a linear or branched alkyl radical containing from 1 to 30 carbon atoms;

4) optionally at least one crosslinking or branching agent; at least one of the monomers of formula (IVa), (IVb) or (VI) comprising at least one fatty chain containing from 8 to 30 carbon atoms and said compounds of the monomers of formulae (IVa), (IVb), (V) and (VI) possibly being quaternized, for example with a C₁-C₄ alkyl halide or a C₁-C₄ dialkyl sulfate.

The monomers of formulae (IVa) and (IVb) of the present invention are preferably chosen from the group formed by:
dimethylaminoethyl methacrylate, dimethylaminoethyl acrylate,
diethylaminoethyl methacrylate, diethylaminoethyl acrylate,
dimethylaminopropyl methacrylate, dimethylaminopropyl acrylate,
dimethylaminopropylmethacrylamide or dimethylaminopropylacrylamide, optionally quaternized, for example with a C₁-C₄ alkyl halide or a C₁-C₄ dialkyl sulfate.

More particularly, the monomer of formula (IVa) is chosen from acrylamidopropyltrimethylammonium chloride and methacrylamidopropyltrimethylammonium chloride.

The compounds of formula (V) of the present invention are preferably chosen from the group formed by acrylic acid, methacrylic acid, crotonic acid, 2-methylcrotonic acid, 2-acrylamido-2-methylpropanesulfonic acid and 2-methacrylamido-2-methylpropanesulfonic acid. More particularly, the monomer of formula (V) is acrylic acid.

The monomers of formula (VI) of the present invention are preferably chosen from the group formed by C₁₂-C₂₂ and more particularly C₁₆-C₁₈ alkyl acrylates or methacrylates.

The crosslinking or branching agent is preferably chosen from N,N′-methylenebisacrylamide, triallylmethylammonium chloride, allyl methacrylate, n-methylolacrylamide, polyethylene glycol dimethacrylates, ethylene glycol dimethacrylate, diethylene glycol dimethacrylate, 1,6-hexanediol dimethacrylate and allyl sucrose.

The polymers according to the invention may also contain other monomers such as nonionic monomers and in particular C₁-C₄ alkyl acrylates or methacrylates.

The ratio of the number of cationic charges/anionic charges in these amphoteric polymers is preferably equal to about 1.

The weight-average molecular weights of the associative amphoteric polymers have a weight-average molecular mass of greater than 500, preferably between 10 000 and 10 000 000 and even more preferentially between 100 000 and 8 000 000.

Preferably, the associative amphoteric polymers of the invention contain from 1 mol % to 99 mol %, more preferentially from 20 mol % to 95 mol % and even more preferentially from 25 mol % to 75 mol % of compound(s) of formula (IVa) or (IVb). They also preferably contain from 1 mol % to 80 mol %, more preferentially from 5 mol % to 80 mol % and even more preferentially from 25 mol % to 75 mol % of compound(s) of formula (V). The content of compound(s) of formula (VI) is preferably between 0.1 mol % and 70 mol %, more preferentially between 1 mol % and 50 mol % and even more preferentially between 1 mol % and 10 mol %. The crosslinking or branching agent, when it is present, is preferably between 0.0001 mol % and 1 mol % and even more preferentially between 0.0001 mol % and 0.1 mol %.

Preferably, the mole ratio between the compound(s) of formula (IVa) or (IVb) and the compound(s) of formula (V) ranges from 20/80 to 95/5 and more preferentially from 25/75 to 75/25.

The associative amphoteric polymers according to the invention are described, for example, in patent application WO 98/44012.

The amphoteric polymers that are particularly preferred according to the invention are chosen from acrylic acid/acrylamidopropyltrimethylammonium chloride/stearyl methacrylate copolymers.

According to a preferred embodiment, the associative polymer is chosen from nonionic associative polymers and more particularly from associative polyurethanes, such as Steareth-100/PEG-136/HDI Copolymer sold under the name Rheolate FX 1100 by Elementis.

Such an associative polymer is advantageously used in a proportion of from 0.1% to 8% by weight of solids and preferably about 3% by weight, relative to the total weight of the aqueous phase.

2. Polyacrylamides and crosslinked and/or neutralized 2-acrylamido-2-methylpropanesulfonic Acid Polymers and Copolymers

The polymers used that are suitable as aqueous gelling agent for the invention may be crosslinked or non-crosslinked homopolymers or copolymers comprising at least the 2-acrylamidomethylpropanesulfonic acid (AMPS®) monomer, in a form partially or totally neutralized with a mineral base other than aqueous ammonia, such as sodium hydroxide or potassium hydroxide.

They are preferably totally or almost totally neutralized, i.e. at least 90% neutralized.

These AMPS® polymers according to the invention may be crosslinked or non-crosslinked.

When the polymers are crosslinked, the crosslinking agents may be chosen from the polyolefinically unsaturated compounds commonly used for crosslinking polymers obtained by free-radical polymerization.

Examples of crosslinking agents that may be mentioned include divinylbenzene, diallyl ether, dipropylene glycol diallyl ether, polyglycol diallyl ethers, triethylene glycol divinyl ether, hydroquinone diallyl ether, ethylene glycol or tetraethylene glycol di(meth)acrylate, trimethylolpropane triacrylate, methylenebisacrylamide, methylenebismethacrylamide, triallylamine, triallyl cyanurate, diallyl maleate, tetraallylethylenediamine, tetraallyloxyethane, trimethylolpropane diallyl ether, allyl (meth)acrylate, allylic ethers of alcohols of the sugar series, or other allyl or vinyl ethers of polyfunctional alcohols, and also the allylic esters of phosphoric and/or vinylphosphonic acid derivatives, or mixtures of these compounds.

According to one preferred embodiment of the invention, the crosslinking agent is chosen from methylenebisacrylamide, allyl methacrylate and trimethylolpropane triacrylate (TMPTA). The degree of crosslinking generally ranges from 0.01 mol % to 10 mol % and more particularly from 0.2 mol % to 2 mol % relative to the polymer.

The AMPS® polymers that are suitable for use in the invention are water-soluble or water-dispersible. They are in this case:
either “homopolymers” comprising only AMPS monomers and, if they are crosslinked, one or more crosslinking agents such as those defined above;
or copolymers obtained from AMPS® and from one or more hydrophilic or hydrophobic ethylenically unsaturated monomers and, if they are crosslinked, one or more crosslinking agents such as those defined above. When said copolymers comprise hydrophobic ethylenically unsaturated monomers, the latter do not comprise a fatty chain and are preferably present in small amounts.

For the purpose of the present invention, the term “fatty chain” is intended to mean any hydrocarbon-based chain containing at least 7 carbon atoms.

The term “water-soluble or water-dispersible” means polymers which, when introduced into an aqueous phase at 25° C., at a mass concentration equal to 1%, make it possible to obtain a macroscopically homogeneous and transparent solution, i.e. a solution with a light maximum transmittance value, at a wavelength equal to 500 nm, through a sample 1 cm thick, of at least 60% and preferably of at least 70%.

The “homopolymers” according to the invention are preferably crosslinked and neutralized, and they may be obtained according to the preparation process comprising the following steps:

(a) the monomer such as AMPS in free form is dispersed or dissolved in a solution of tert-butanol or of water and tert-butanol;

(b) the monomer solution or dispersion obtained in (a) is neutralized with one or more mineral or organic bases, preferably aqueous ammonia NH₃, in an amount making it possible to obtain a degree of neutralization of the sulfonic acid functions of the polymer ranging from 90% to 100%;

(c) the crosslinking monomer(s) are added to the solution or dispersion obtained in (b);

(d) a standard free-radical polymerization is performed in the presence of free-radical initiators at a temperature ranging from 10 to 150° C.; the polymer precipitates in the tert-butanol-based solution or dispersion.

The water-soluble or water-dispersible AMPS® copolymers according to the invention contain water-soluble ethylenically unsaturated monomers, hydrophobic monomers, or mixtures thereof.

The water-soluble co-monomers may be ionic or nonionic.

Among the ionic water-soluble co-monomers, mention may be made, for example, of the following compounds and salts thereof:
(meth)acrylic acid,
styrenesulfonic acid,
vinylsulfonic acid and (meth)allylsulfonic acid,
vinylphosphonic acid,
maleic acid,
itaconic acid,
crotonic acid,
water-soluble vinyl monomers of formula (A) below:

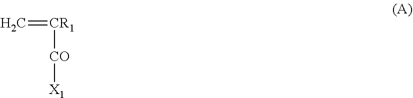

in which:
R, is chosen from H, —CH₃, —C₂H₅ and —C₃H₇;
X₁ is chosen from:
alkyl oxides of type —OR₂ where R₂ is a linear or branched, saturated or unsaturated hydrocarbon-based radical containing from 1 to 6 carbon atoms, substituted with at least one sulfonic (—SO₃—) and/or sulfate (—SO₄—) and/or phosphate (—PO₄H₂—) group.

Among the nonionic water-soluble co-monomers, mention may be made, for example, of:
(meth)acrylamide,
N-vinylacetamide and N-methyl-N-vinylacetamide,
N-vinylformamide and N-methyl-N-vinylformamide,
maleic anhydride,
vinylamine,
N-vinyllactams comprising a cyclic alkyl group containing from 4 to 9 carbon atoms, such as N-vinylpyrrolidone, N-butyrolactam and N-vinylcaprolactam,
vinyl alcohol of formula CH₂═CHOH,
water-soluble vinyl monomers of formula (B) below:

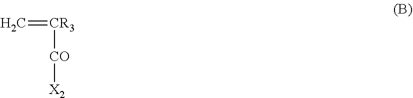

in which:
R₃ is chosen from H, —CH₃, —C₂H₅ and —C₃H—;
X₂ is chosen from:
alkyl oxides of the type —OR₄ where R₄ is a linear or branched, saturated or unsaturated hydrocarbon-based radical having from 1 to 6 carbon atoms, optionally substituted with a halogen (iodine, bromine, chlorine or fluorine) atom; a hydroxyl (—OH) group; ether.

Mention may be made, for example, of glycidyl (meth)acrylate, hydroxyethyl methacrylate, and (meth)acrylates of ethylene glycol, of diethylene glycol or of polyalkylene glycol.

Among the hydrophobic comonomers without a fatty chain, mention may be made, for example, of:
styrene and derivatives thereof, such as 4-butylstyrene, α-methylstyrene and vinyltoluene;
vinyl acetate of formula CH₂—CH—OCOCH₃;
vinyl ethers of formula CH₂═CHOR in which R is a linear or branched, saturated or unsaturated hydrocarbon-based radical containing from 1 to 6 carbons;
acrylonitrile;
caprolactone;
vinyl chloride and vinylidene chloride;
silicone derivatives, which, after polymerization, result in silicone polymers such as methacryloxypropyltris(trimethylsiloxy)silane and silicone methacrylamides;
hydrophobic vinyl monomers of formula (C) below:

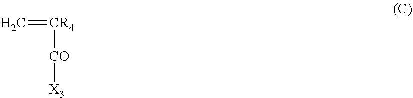

in which:
R₄ is chosen from H, —CH₃, —C₂H₅ and —C₃H₇;
X₃ is chosen from:
alkyl oxides of the type —OR₅ where R₅ is a linear or branched, saturated or unsaturated hydrocarbon-based radical containing from 1 to 6 carbon atoms.

Mention may be made, for example, of methyl methacrylate, ethyl methacrylate, n-butyl (meth)acrylate, tert-butyl (meth)acrylate, cyclohexyl acrylate, isobornyl acrylate and 2-ethylhexyl acrylate.

The water-soluble or water-dispersible AMPS® polymers of the invention preferably have a molar mass ranging from 50 000 g/mol to 10 000 000 g/mol, preferably from 80 000 g/mol to 8 000 000 g/mol, and even more preferably from 100 000 g/mol to 7 000 000 g/mol.

As water-soluble or water-dispersible AMPS homopolymers in accordance with the invention, mention may be made, for example, of crosslinked or non-crosslinked polymers of sodium acrylamido-2-methylpropanesulfonate, such as that used in the commercial product Simulgel 800 (CTFA name: Sodium Polyacryloyldimethyl Taurate), crosslinked ammonium acrylamido-2-methylpropanesulfonate polymers (INCI name: Ammonium polydimethyltauramide) such as those described in patent EP 0 815 928 B 1 and such as the product sold under the trade name Hostacerin AMPS® by the company Clariant.

As water-soluble or water-dispersible AMPS copolymers in accordance with the invention, examples that may be mentioned include:
crosslinked acrylamide/sodium acrylamido-2-methylpropanesulfonate copolymers, such as that used in the commercial product Sepigel 305 (CTFA name: Polyacrylamide/C₁₃-C₁₄ Isoparaffin/Laureth-7) or that used in the commercial product sold under the name Simulgel 600 (CTFA name: Acrylamide/Sodium Acryloyldimethyltaurate/Isohexadecane/Polysorbate-80) by the company SEPPIC;
copolymers of AMPS® and of vinylpyrrolidone or vinylformamide, such as that used in the commercial product sold under the name Aristoflex AVC® by the company Clariant (CTFA name: Ammonium acryloyldimethyltaurate/VP copolymer) but neutralized with sodium hydroxide or potassium hydroxide;
copolymers of AMPS® and of sodium acrylate, for instance the AMPS/sodium acrylate copolymer, such as that used in the commercial product sold under the name Simulgel EG® by the company SEPPIC or under the trade name Sepinov EM (CTFA name: Hydroxyethyl acrylate/sodium acryloyldimethyltaurate copolymer);
copolymers of AMPS® and of hydroxyethyl acrylate, for instance the AMPS®/hydroxyethyl acrylate copolymer, such as that used in the commercial product sold under the name Simulgel NS® by the company SEPPIC (CTFA name: Hydroxyethyl acrylate/sodium acryloyldimethyltaurate copolymer (and) squalane (and) polysorbate 60), or such as the product sold under the name Sodium acrylamido-2-methylpropanesulfonate/hydroxyethyl acrylate copolymer, such as the commercial product Sepinov EMT 10 (INCI name: Hydroxyethyl acrylate/sodium acryloyldimethyltaurate copolymer).

Preferably, the product sold under the name Sodium acrylamido-2-methylpropanesulfonate/hydroxyethyl acrylate copolymer, such as the commercial product Sepinov EMT 10 (INCI name: Hydroxyethyl acrylate/sodium acryloyldimethyl taurate copolymer) is used as water-soluble or water-dispersible AMPS copolymers in accordance with the invention.

In general, an aqueous phase according to the invention may comprise from 0.1% to 8% by weight of solids, preferably 0.2% to 5% by weight and more preferentially from 0.7% to 2.5% by weight of polyacrylamide(s) and/or of crosslinked and/or neutralized 2-acrylamido-2-methylpropanesulfonic acid polymer(s) and copolymer(s) relative to its total weight.

3. Modified or Unmodified Carboxyvinyl Polymers

The modified or unmodified carboxyvinyl polymers may be copolymers derived from the polymerization of at least one monomer (a) chosen from α,β-ethylenically unsaturated carboxylic acids or esters thereof, with at least one ethylenically unsaturated monomer (b) comprising a hydrophobic group.

The term “copolymers” means both copolymers obtained from two types of monomer and those obtained from more than two types of monomer, such as terpolymers obtained from three types of monomer.

Their chemical structure more particularly comprises at least one hydrophilic unit and at least one hydrophobic unit. The term “hydrophobic group or unit” means a radical with a saturated or unsaturated, linear or branched hydrocarbon-based chain, comprising at least 8 carbon atoms, preferably from 10 to 30 carbon atoms, in particular from 12 to 30 carbon atoms and more preferentially from 18 to 30 carbon atoms.

Preferably, these copolymers are chosen from copolymers derived from the polymerization:
of at least one monomer of formula (1) below:

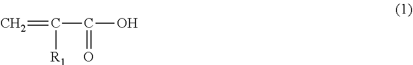

in which R₁ denotes H or CH₃ or C₂H₅, i.e. acrylic acid, methacrylic acid or ethacrylic acid monomers, and
of at least one monomer of unsaturated carboxylic acid (C₁₀-C₃₀)alkyl ester type corresponding to the monomer of formula (2) below:

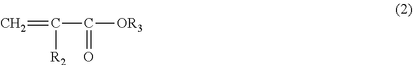

in which R₂ denotes H or CH₃ or C₂H₃ (i.e. acrylate, methacrylate or ethacrylate units) and preferably H (acrylate units) or CH₃ (methacrylate units), R₃ denoting a C₁₀-C₃₀ and preferably C₁₂-C₂₂ alkyl radical.

The unsaturated carboxylic acid (C₁₀-C₃₀)alkyl esters are preferably chosen from lauryl acrylate, stearyl acrylate, decyl acrylate, isodecyl acrylate and dodecyl acrylate, and the corresponding methacrylates, such as lauryl methacrylate, stearyl methacrylate, decyl methacrylate, isodecyl methacrylate and dodecyl methacrylate, and mixtures thereof.

According to a preferred embodiment, these polymers are crosslinked.

Among the copolymers of this type, use will more particularly be made of polymers derived from the polymerization of a monomer mixture comprising:
essentially acrylic acid,
an ester of formula (2) described above in which R₂ denotes H or CH₃, R₃ denoting an alkyl radical containing from 12 to 22 carbon atoms,

(iii) and a crosslinking agent, which is a well-known copolymerizable polyethylenic unsaturated monomer, such as diallyl phthalate, allyl (meth)acrylate, divinylbenzene, (poly)ethylene glycol dimethacrylate or methylenebisacrylamide.

Among the copolymers of this type, use will more particularly be made of those consisting of from 95% to 60% by weight of acrylic acid (hydrophilic unit), 4% to 40% by weight of C₁₀-C₃₀ alkyl acrylate (hydrophobic unit) and 0% to 6% by weight of crosslinking polymerizable monomer, or alternatively those consisting of from 98% to 96% by weight of acrylic acid (hydrophilic unit), 1% to 4% by weight of C₁₀-C₃₀ alkyl acrylate (hydrophobic unit) and 0.1% to 0.6% by weight of crosslinking polymerizable monomer such as those described previously.

Among the abovementioned polymers, the ones that are most particularly preferred according to the present invention are acrylate/C₁₀-C₃₀-alkyl acrylate copolymers (INCI name: Acrylates/C₁₀-C₃₀ Alkyl acrylate Crosspolymer) such as the products sold by the company Lubrizol under the trade names Pemulen TR-1, Pemulen TR-2, Carbopol 1382, Carbopol EDT 2020 and Carbopol Ultrez 20 Polymer, and even more preferentially Pemulen TR-2.

Among the modified or unmodified carboxyvinyl polymers, mention may also be made of sodium polyacrylates such as those sold under the name Cosmedia SP® containing 90% solids and 10% water, or Cosmedia SPL® as an inverse emulsion containing about 60% solids, an oil (hydrogenated polydecene) and a surfactant (PPG-5 Laureth-5), both sold by the company Cognis.

Mention may also be made of partially neutralized sodium polyacrylates that are in the form of an inverse emulsion comprising at least one polar oil, for example the product sold under the name Luvigel® EM by the company BASF.

The modified or unmodified carboxyvinyl polymers may also be chosen from crosslinked (meth)acrylic acid homopolymers.

For the purposes of the present patent application, the term “(meth)acrylic” means “acrylic or methacrylic”.

Examples that may be mentioned include the products sold by Lubrizol under the names Carbopol 910, 934, 940, 941, 934 P, 980, 981, 2984, 5984 and Carbopol Ultrez 10 Polymer, or by 3V-Sigma under the name Synthalen® K, Synthalen® L or Synthalen® M.

Among the modified or unmodified carboxyvinyl polymers, mention may be made in particular of Carbopol (CTFA name: carbomer) and Pemulen (CTFA name: Acrylates/C 10-30 alkyl acrylate crosspolymer) sold by the company Lubrizol.

The modified or unmodified carboxyvinyl polymers may be present in a proportion of from 0.1% to 5% by weight of solids relative to the weight of the aqueous phase, in particular from 0.3% to 1% by weight and preferably in a proportion of about 1% by weight, relative to the weight of the aqueous phase.

Advantageously, the hydrophilic gelling agent is at least one synthetic polymeric gelling agent chosen from crosslinked acrylic homopolymers or copolymers; polyacrylamides and crosslinked and/or neutralized 2-acrylamido-2-methylpropanesulfonic acid polymers and copolymers; modified or unmodified carboxyvinyl polymers, and mixtures thereof.

More particularly, it is at least a 2-acrylamido-2-methylpropanesulfonic acid polymer or copolymer, an associative polyurethane and/or a crosslinked sodium polyacrylate.

### III. Other Hydrophilic Gelling Agents

These gelling agents are more particularly chosen from mixed silicates and fumed silicas.

III.A Mixed Silicate

For the purposes of the present invention, the term “mixed silicate” means any silicate of natural or synthetic origin containing several (two or more) types of cations chosen from alkali metals (for example Na, Li, K) or alkaline-earth metals (for example Be, Mg, Ca), transition metals and aluminium.

According to a particular embodiment, the mixed silicate(s) are in the form of solid particles containing at least 10% by weight of at least one silicate relative to the total weight of the particles. In the rest of the present description, these particles will be referred to as “silicate particles”.

Preferably, the silicate particles contain less than 1% by weight of aluminium relative to the total weight of the particles. Even more preferably, they contain from 0% to 1% by weight of aluminium relative to the total weight of the particles.

Preferably, the silicate particles contain at least 50% by weight and better still at least 70% by weight of the silicate relative to the total weight of the particles. Particles containing at least 90% by weight of silicates, relative to the total weight of the particles, are particularly preferred.

In particular, this is a silicate or a mixture of silicates and of alkali metals or alkaline-earth metals, of aluminium or of iron.

Preferably, it is sodium, magnesium and/or lithium silicate.

To ensure good cosmetic properties, these silicates are generally in finely divided form, and in particular in the form of particles with a mean size ranging from 2 nm to 1 μm (from 2 nm to 1000 nm), preferably from 5 nm to 600 nm and even more preferentially from 20 to 250 nm.

Silicate particles may have any form, for example the form of spheres, flakes, needles, platelets, disks or leaflets, or totally random forms. Preferably, the silicate particles have the form of disks or leaflets.

Also, the term “mean size” of the particles means the number-average size of the largest dimension (length) that it is possible to measure between two diametrically opposite points on an individual particle. The size may be determined, for example, by transmission electron microscopy or by measuring the specific surface area by the BET method or alternatively by means of a laser particle sizer.

When the particles are in the form of disks or leaflets, they generally have a thickness ranging from about 0.5 nm to 5 nm.

The silicate particles may consist of an alloy with metal or metalloid oxides, obtained, for example, via thermal fusion of its various constituents. When the particles also comprise such a metal or metalloid oxide, it is preferably chosen from silicon, boron or aluminium oxide.

According to a particular embodiment of the invention, the silicates are phyllosilicates, i.e. silicates having a structure in which the SiO₄ tetrahedra are organized as leaflets between which the metal cations are enclosed.

The mixed silicates that are suitable for use in the invention may be chosen, for example, from montmorillonites, hectorites, bentonites, beidellite and saponites. According to a preferred embodiment of the invention, the mixed silicates used are more particularly chosen from hectorites and bentonites, and better still from laponites.

A family of silicates that is particularly preferred in the compositions of the present invention is thus that of laponites. Laponites are sodium magnesium silicates also possibly containing lithium, which have a layer structure similar to that of montmorillonites. Laponite is the synthetic form of the natural mineral known as hectorite. The synthetic origin of this family of silicates is of considerable advantage over the natural form, since it allows good control of the composition of the product. In addition, laponites have the advantage of having a particle size that is much smaller than that of the natural minerals hectorite and bentonite.

Laponites that may especially be mentioned include the products sold under the following names: Laponite® XLS, Laponite® XLG, Laponite® RD, Laponitee RDS and Laponite® XL21 (these products are sodium magnesium silicates and sodium lithium magnesium silicates) by the company Rockwood Additives Limited.

Such gelling agents may be used in a proportion of from 0.1% to 8% by weight of solids relative to the total weight of the aqueous phase, especially from 0.1% to 5% by weight and in particular from 0.5% to 3% by weight, relative to the total weight of the aqueous phase.

III.B Hydrophilic Fumed Silica

The fumed silicas according to the present invention are hydrophilic.

Hydrophilic fumed silicas are obtained by pyrolysis of silicon tetrachloride (SiCl₄) in a continuous flame at 1000° C. in the presence of hydrogen and oxygen. Among the fumed silicas of hydrophilic nature that may be used according to the present invention, mention may be made especially of the products sold by the company Degussa or Evonik-Degussa under the trade names Aerosil® 90, 130, 150, 200, 300 and 380 or by the company Cabot under the name Carbosil H5.

Such gelling agents may be used in a proportion of from 0.1% to 10% by weight of solids relative to the total weight of the aqueous phase, especially from 0.1% to 5% by weight and in particular from 0.5% to 3% by weight, relative to the total weight of the aqueous phase.

### Lipophilic Gelling Agent

For the purposes of the present invention, the term “lipophilic gelling agent” means a compound that is capable of gelling the oily phase of the compositions according to the invention.

The gelling agent is lipophilic and is thus present in the oily phase of the composition.

The gelling agent is liposoluble or lipodispersible.

As emerges from the foregoing, the lipophilic gelling agent is advantageously chosen from hydrogen bonding polymers and more particularly polyamides, and mixtures thereof.

### Polyamides

The oily phase of a composition according to the invention may comprise at least one polyamide chosen from hydrocarbon-based polyamides and silicone polyamides, and mixtures thereof.

This type of gelling agent is particularly advantageous with regard to the gloss and colour-revealing effect it affords when it is combined with a dyestuff.

It is therefore particularly advantageous for formulating cosmetic compositions intended for making up the skin and the lips.

Preferably, the total content of polyamide(s) is between 0.1% and 30% by weight expressed as solids, preferably between 0.1% and 20% by weight and preferably between 0.5% and 10% by weight relative to the total weight of the oily phase.

For the purposes of the invention, the term “polyamide” means a compound containing at least two repeating amide units, preferably at least three repeating amide units and better still ten repeating amide units.

A. Hydrocarbon-Based Polyamide

The term “hydrocarbon-based polyamide” means a polyamide formed essentially of, indeed even consisting of, carbon and hydrogen atoms, and optionally of oxygen or nitrogen atoms, and not comprising any silicon or fluorine atoms. It may contain alcohol, ester, ether, carboxylic acid, amine and/or amide groups.

For the purposes of the invention, the term “functionalized chain” means an alkyl chain comprising one or more functional groups or reagents chosen especially from hydroxyl, ether, ester, oxyalkylene and polyoxyalkylene groups.

Advantageously, this polyamide of the composition according to the invention has a weight-average molecular mass of less than 100 000 g/mol (especially ranging from 1000 to 100 000 g/mol), in particular less than 50 000 g/mol (especially ranging from 1000 to 50 000 g/mol) and more particularly ranging from 1000 to 30 000 g/mol, preferably from 2000 to 20 000 g/mol and better still from 2000 to 10 000 g/mol.

This polyamide is insoluble in water, in particular at 25° C.

According to a first embodiment of the invention, the polyamide used is a polyamide of formula (I):

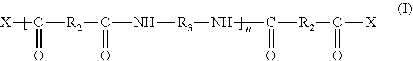

in which X represents a group —N(R₁)₂ or a group —OR₁ in which R₁ is a linear or branched C₈ to C₂₂ alkyl radical which may be identical or different, R₂ is a C₂₈-C₄₂ diacid dimer residue, R₃ is an ethylenediamine radical and n is between 2 and 5;
and mixtures thereof.

According to a particular mode, the polyamide used is an amide-terminated polyamide of formula (Ia):

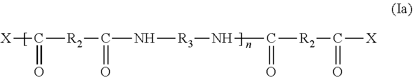

in which X represents a group —N(R₁)₂ in which R₁ is a linear or branched C₈ to C₂₂ alkyl radical which may be identical or different, R₂ is a C₂₈-C₄₂ diacid dimer residue, R₃ is an ethylenediamine radical and n is between 2 and 5;
and mixtures thereof.

The oily phase of a composition according to the invention may also comprise, additionally in this case, at least one additional polyamide of formula (Ib):

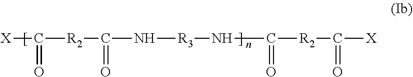

in which X represents a group —OR₁ in which R₁ is a linear or branched C₈ to C₂₂ and preferably C₁₆ to C₂₂ alkyl radical which may be identical or different, R₂ is a C₂₈-C₄₂ diacid dimer residue, R₃ is an ethylenediamine radical and n is between 2 and 5, mention may be made of the commercial products sold by the company Arizona Chemical under the names Uniclear 80 and Uniclear 100 or Uniclear 80 V, Uniclear 100 V and Uniclear 100 VG, the INCI name of which is Ethylenediamine/stearyl dimer dilinoleate copolymer.

B. Silicone Polyamide

The silicone polyamides are preferably solid at room temperature (25° C.) and atmospheric pressure (760 mmHg).

The silicone polyamides may preferentially be polymers comprising at least one unit of formula (III) or (IV):

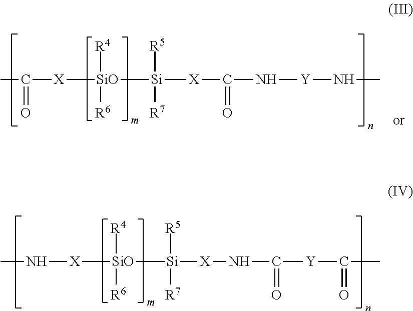

in which:
R⁴, R⁵, R⁶ and R⁷, which may be identical or different, represent a group chosen from:
saturated or unsaturated, C₁ to C₄₀ linear, branched or cyclic hydrocarbon-based groups, which may contain in their chain one or more oxygen, sulfur and/or nitrogen atoms, and which may be partially or totally substituted with fluorine atoms,
C₆ to C₁₀ aryl groups, optionally substituted with one or more C₁ to C₄ alkyl groups,
polyorganosiloxane chains possibly containing one or more oxygen, sulfur and/or nitrogen atoms,
the groups X, which may be identical or different, represent a linear or branched C₁ to C₃₀ alkylenediyl group, possibly containing in its chain one or more oxygen and/or nitrogen atoms,
Y is a saturated or unsaturated C₁ to C₅₀ linear or branched alkylene, arylene, cycloalkylene, alkylarylene or arylalkylene divalent group, which may comprise one or more oxygen, sulfur and/or nitrogen atoms, and/or may bear as substituent one of the following atoms or groups of atoms: fluorine, hydroxyl, C₃ to C₈ cycloalkyl, C₁ to C₄₀ alkyl, C₅ to C₁₀ aryl, phenyl optionally substituted with one to three C₁ to C₃ alkyl, C₁ to C₃ hydroxyalkyl and C₁ to C₆ aminoalkyl groups, or Y represent a group corresponding to the formula:

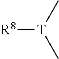

in which:
T represents a linear or branched, saturated or unsaturated, C₃ to C₂₄ trivalent or tetravalent hydrocarbon-based group optionally substituted with a polyorganosiloxane chain, and possibly containing one or more atoms chosen from O, N and S, or T represents a trivalent atom chosen from N, P and Al, and
R8 represents a linear or branched C₁-C₅₀ alkyl group or a polyorganosiloxane chain, possibly comprising one or more ester, amide, urethane, thiocarbamate, urea, thiourea and/or sulfonamide groups, which may possibly be linked to another chain of the polymer,
n is an integer ranging from 2 to 500 and preferably from 2 to 200, and m is an integer ranging from 1 to 1000, preferably from 1 to 700 and even better still from 6 to 200.

According to a particular mode, the silicone polyamide comprises at least one unit of formula (III) in which m ranges from 50 to 200, in particular from 75 to 150 and is preferably about 100.

More preferably, R⁴, R⁵, R⁶ and R⁷ independently represent a linear or branched C₁ to C₄₀ alkyl group, preferably a group CH₃, C₂H5, n-C₃H₇ or isopropyl in formula (III).

As examples of silicone polymers that may be used, mention may be made of one of the silicone polyamides obtained in accordance with Examples 1 to 3 of document U.S. Pat. No. 5,981,680.

Mention may be made of the compounds sold by the company Dow Corning under the name DC 2-8179 (DP 100) and DC 2-8178 (DP 15), the INCI name of which is Nylon-611/dimethicone copolymers, i.e. Nylon-611/dimethicone copolymers. The silicone polymers and/or copolymers advantageously have a temperature of transition from the solid state to the liquid state ranging from 45° C. to 190° C. Preferably, they have a temperature of transition from the solid state to the liquid state ranging from 70 to 130° C. and better still from 80° C. to 105° C.

Preferably, the total content of polyamide(s) and/or silicone polyamide(s) is between 0.5% and 25% by weight of solids, in particular from 2% to 20% by weight and preferably between 2% and 12% by weight relative to the total weight of the oily phase.

Advantageously, the hydrogen bonding polymer is chosen from ethylenediamine/stearyl dimer dilinoleate copolymer and Nylon-611/dimethicone copolymers.

### Hydrophilic Gelling Agent(S)/Lipophilic Gelling Agent(S) Systems

As non-limiting illustrations of hydrophilic gelling agent(s)/lipophilic gelling agent(s) systems that are most particularly suitable for use in the invention, mention may be made of the system of synthetic polymeric hydrophilic gelling agent(s)/hydrogen bonding polymer(s).

As preferred hydrophilic gelling agents of synthetic polymeric type that are suitable for use in the invention, mention may be made more particularly of 2-acrylamido-2-methylpropanesulfonic acid polymers, for instance AMPS and 2-acrylamido-2-methylpropanesulfonic acid copolymers, 2-acrylamido-2-methylpropanesulfonic acid polymers, for instance AMPS and 2-acrylamido-2-methylpropanesulfonic acid copolymers and in particular copolymers of AMPS® and of hydroxyethyl acrylate, for instance the AMPS®/hydroxyethyl acrylate copolymer, such as that used in the commercial product sold under the name Simulgel NS® by the company SEPPIC (CTFA name: Hydroxyethyl acrylate/sodium acryloyldimethyltaurate copolymer (and) squalane (and) polysorbate 60), or such as the product sold under the name Sodium acrylamido-2-methylpropanesulfonate/hydroxyethyl acrylate copolymer, such as the commercial product Sepinov EMT 10 (INCI name: Hydroxyethyl acrylate/sodium acryloyldimethyltaurate copolymer).

As preferred lipophilic hydrogen bonding polymers that are suitable for use in the invention, mention may be made more particularly of hydrocarbon-based polyamides, for instance the commercial products sold by the company Arizona Chemical under the names Uniclear 80 and Uniclear 100 or Uniclear 80 V, Uniclear 100 V and Uniclear 100 VG, the INCI name of which is ethylenediamine/stearyl dimer dilinoleate copolymer, and silicone polyamides, for instance the compounds sold by the company Dow Corning under the name DC 2-8179 (DP 100) and DC 2-8178 (DP 15), the INCI name of which is Nylon-611/dimethicone copolymers, i.e. Nylon-611/dimethicone copolymers.

Thus, a composition according to the invention may advantageously comprise as hydrophilic gelling agent(s)/lipophilic gelling agent(s) systems, a system of the type:
copolymer of 2-acrylamido-2-methylpropanesulfonic acid and of hydroxyethyl acrylate/hydrocarbon-based polyamide; or
copolymer of 2-acrylamido-2-methylpropanesulfonic acid and of hydroxyethyl acrylate/silicone polyamide.

In particular, it is a system of the type:
copolymer of 2-acrylamido-2-methylpropanesulfonic acid and of hydroxyethyl acrylate/ethylenediamine/stearyl dimer dilinoleate copolymer; and
copolymer of 2-acrylamido-2-methylpropanesulfonic acid and of hydroxyethyl acrylate/Nylon-611/dimethicone copolymer.

### Aqueous Phase

The aqueous phase of a composition according to the invention comprises water and optionally a water-soluble solvent.

In the present invention, the term “water-soluble solvent” denotes a compound that is liquid at room temperature and water-miscible (miscibility with water of greater than 50% by weight at 25° C. and atmospheric pressure).

The water-soluble solvents that may be used in the composition of the invention may also be volatile.

Among the water-soluble solvents that may be used in the composition in accordance with the invention, mention may be made especially of lower monoalcohols containing from 1 to 5 carbon atoms, such as ethanol and isopropanol, glycols containing from 2 to 8 carbon atoms, such as ethylene glycol, propylene glycol, 1,3-butylene glycol and dipropylene glycol, C₃ and C₄ ketones and C₂-C₄ aldehydes.

The aqueous phase (water and optionally the water-miscible solvent) may be present in the composition in a content ranging from 5% to 95%, better still from 30% to 80% by weight and preferably from 40% to 75% by weight relative to the total weight of the said composition.

According to another embodiment variant, the aqueous phase of a composition according to the invention may comprise at least one C₂-C₃₂ polyol.

For the purposes of the present invention, the term “polyol” should be understood as meaning any organic molecule comprising at least two free hydroxyl groups.

Preferably, a polyol in accordance with the present invention is present in liquid form at room temperature.

A polyol that is suitable for use in the invention may be a compound of linear, branched or cyclic, saturated or unsaturated alkyl type, bearing on the alkyl chain at least two —OH functions, in particular at least three —OH functions and more particularly at least four —OH functions.

The polyols advantageously suitable for the formulation of a composition according to the present invention are those exhibiting in particular from 2 to 32 carbon atoms and preferably from 3 to 16 carbon atoms.

Advantageously, the polyol may be chosen, for example, from ethylene glycol, pentaerythritol, trimethylolpropane, propylene glycol, 1,3-propanediol, butylene glycol, isoprene glycol, pentylene glycol, hexylene glycol, glycerol, polyglycerols such as glycerol oligomers, for instance diglycerol, and polyethylene glycols, and mixtures thereof.

According to a preferred embodiment of the invention, the said polyol is chosen from ethylene glycol, pentaerythritol, trimethylolpropane, propylene glycol, glycerol, polyglycerols and polyethylene glycols, and mixtures thereof.

According to a particular embodiment, the composition of the invention may comprise at least propylene glycol.

According to another particular embodiment, the composition of the invention may comprise at least glycerol.

### Oily Phase

For the purposes of the invention, an oily phase comprises at least one oil.

The term “oil” means any fatty substance that is in liquid form at room temperature and atmospheric pressure.

An oily phase that is suitable for preparing the cosmetic compositions according to the invention may comprise hydrocarbon-based oils, silicone oils, fluoro oils or non-fluoro oils, or mixtures thereof.

The oils may be volatile or non-volatile.

They can be of animal, vegetable, mineral or synthetic origin. According to one embodiment variant, oils of plant origin are preferred.

For the purposes of the present invention, the term “non-volatile oil” means an oil with a vapour pressure of less than 0.13 Pa.

For the purposes of the present invention, the term “silicone oil” is intended to mean an oil comprising at least one silicon atom, and in particular at least one Si—O group.

The term “fluoro oil” means an oil comprising at least one fluorine atom.

The term “hydrocarbon-based oil” means an oil mainly containing hydrogen and carbon atoms.

The oils may optionally comprise oxygen, nitrogen, sulfur and/or phosphorus atoms, for example in the form of hydroxyl or acid radicals.

For the purposes of the invention, the term “volatile oil” means any oil that is capable of evaporating on contact with the skin in less than one hour, at room temperature and atmospheric pressure. The volatile oil is a volatile cosmetic compound, which is liquid at room temperature, especially having a nonzero vapour pressure, at room temperature and atmospheric pressure, in particular having a vapour pressure ranging from 0.13 Pa to 40 000 Pa (10⁻³ to 300 mmHg), in particular ranging from 1.3 Pa to 13 000 Pa (0.01 to 100 mmHg) and more particularly ranging from 1.3 Pa to 1,300 Pa (0.01 to 10 mmHg).

Volatile Oils

The volatile oils may be hydrocarbon-based oils or silicone oils.

Among the volatile hydrocarbon-based oils containing from 8 to 16 carbon atoms, mention may be made especially of branched C₈-C₁₆ alkanes, for instance C₈-C₁₆ isoalkanes (also known as isoparaffins), isododecane, isodecane, isohexadecane and, for example, the oils sold under the trade names Isopar or Permethyl, branched C₈-C₁₆ esters, for instance isohexyl neopentanoate, and mixtures thereof. Preferably, the volatile hydrocarbon-based oil is chosen from volatile hydrocarbon-based oils containing from 8 to 16 carbon atoms, and mixtures thereof, in particular from isododecane, isodecane and isohexadecane, and is especially isohexadecane.

Mention may also be made of volatile linear alkanes comprising from 8 to 16 carbon atoms, in particular from 10 to 15 carbon atoms and more particularly from 11 to 13 carbon atoms, for instance n-dodecane (C12) and n-tetradecane (C14) sold by Sasol under the respective references Parafol 12-97 and Parafol 14-97, and also mixtures thereof, the undecane-tridecane mixture, mixtures of n-undccane (C11) and of n-tridecane (C13) obtained in Examples 1 and 2 of patent application WO 2008/155 059 from the company Cognis, and mixtures thereof.

Volatile silicone oils that may be mentioned include linear volatile silicone oils such as hexamethyldisiloxane, octamethyltrisiloxane, decamethyltetrasiloxane, tetradecamethylhexasiloxane, hexadecamethylheptasiloxane and dodecamethylpentasiloxane.

Volatile cyclic silicone oils that may be mentioned include hexamethylcyclotrisiloxane, octamethylcyclotetrasiloxane, decamethylcyclopentasiloxane and dodecamethylcyclohexasiloxane.

Non-Volatile Oils

The non-volatile oils may be chosen especially from non-volatile hydrocarbon-based, fluoro and/or silicone oils.

Non-volatile hydrocarbon-based oils that may especially be mentioned include:
hydrocarbon-based oils of animal origin,
hydrocarbon-based oils of plant origin, synthetic ethers containing from 10 to 40 carbon atoms, such as dicaprylyl ether,
synthetic esters, for instance the oils of formula R₁COOR₂, in which R₁ represents a linear or branched fatty acid residue containing from 1 to 40 carbon atoms and R₂ represents a hydrocarbon-based chain, which is especially branched, containing from 1 to 40 carbon atoms, on condition that R₁+R₂ z 10. The esters may be chosen especially from fatty alcohol and fatty acid esters, for instance cetostearyl octanoate, isopropyl alcohol esters such as isopropyl myristate or isopropyl palmitate, ethyl palmitate, 2-ethylhexyl palmitate, isopropyl stearate, octyl stearate, hydroxylated esters, for instance isostearyl lactate or octyl hydroxystearate, alkyl or polyalkyl ricinoleates, hexyl laurate, neopentanoic acid esters, for instance isodecyl neopentanoate or isotridecyl neopentanoate, and isononanoic acid esters, for instance isononyl isononanoate or isotridecyl isononanoate,
polyol esters and pentaerythritol esters, for instance dipentaerythrityl tetrahydroxystearate/tetraisostearate,
fatty alcohols that are liquid at room temperature, with a branched and/or unsaturated carbon-based chain containing from 12 to 26 carbon atoms, for instance 2-octyldodecanol, isostearyl alcohol and oleyl alcohol,
C₁₂-C₂₂ higher fatty acids, such as oleic acid, linoleic acid, linolenic acid, and mixtures thereof,
non-phenyl silicone oils, for instance caprylyl methicone, and
phenyl silicone oils, for instance phenyl trimethicones, phenyl dimethicones, phenyltrimethylsiloxydiphenylsiloxanes, diphenyl dimethicones, diphenylmethyldiphenyltrisiloxanes and 2-phenylethyl trimethylsiloxysilicates, dimethicones or phenyl trimethicone with a viscosity of less than or equal to 100 cSt, and trimethyl pentaphenyl trisiloxane, and mixtures thereof; and also mixtures of these various oils.

Preferably, a composition according to the invention comprises volatile and/or non volatile silicone oils. Such silicone oils are particularly appreciated when the oily gelling agent is an organopolysiloxane polymer.

A composition according to the invention may comprise from 5% to 95% by weight, better still from 5% to 40% by weight and preferably from 7% to 35% by weight of oil(s) relative to the total weight of the said composition.

As mentioned above, the gelled oily phase according to the invention may have a threshold stress of greater than 1.5 Pa and preferably greater than 10 Pa. This threshold stress value reflects a gel-type texture of this oily phase.

### Dyestuffs

A composition according to the invention may also comprise at least one particulate or non-particulate, water-soluble or water-insoluble dyestuff, preferably in a proportion of at least 0.01% by weight relative to the total weight of the composition.

For obvious reasons, this amount is liable to vary significantly with regard to the intensity of the desired colour effect and of the colour intensity afforded by the dyestuffs under consideration, and its adjustment clearly falls within the competence of a person skilled in the art.

A composition according to the invention may comprise from 0.01% to 15% by weight, especially from 0.1% to 15% by weight, in particular from 1% to 15% by weight and preferably from 5% to 15% by weight of dyestuffs relative to the total weight of the said composition. As stated above, the dyestuffs that are suitable for use in the invention may be water-soluble, but may also be liposoluble.

Advantageously, a composition according to the invention may comprise from 0.01% to 25% by weight, especially from 0.1% to 25% by weight, in particular from 1% to 20% by weight and preferably from 5% to 15% by weight of dyestuffs relative to the total weight of the said composition.

For the purposes of the invention, the term “water-soluble dyestuff” means any natural or synthetic, generally organic compound, which is soluble in an aqueous phase or in water-miscible solvents, and which is capable of imparting colour.

As water-soluble dyes that are suitable for use in the invention, mention may be made especially of synthetic or natural water-soluble dyes, for instance FDC Red 4, DC Red 6, DC Red 22, DC Red 28, DC Red 30, DC Red 33, DC Orange 4, DC Yellow 5, DC Yellow 6, DC Yellow 8, FDC Green 3, DC Green 5, FDC Blue 1, betanin (beetroot), carmine, copper chlorophylline, methylene blue, anthocyanins (enocianin, black carrot, hibiscus and elder), caramel and riboflavin.

The water-soluble dyes are, for example, beetroot juice and caramel.

For the purposes of the invention, the term “liposoluble dyestuff” means any natural or synthetic, generally organic compound, which is soluble in an oily phase or in solvents that are miscible with a fatty substance, and which is capable of imparting colour.

As liposoluble dyes that are suitable for use in the invention, mention may be made especially of synthetic or natural liposoluble dyes, for instance DC Red 17, DC Red 21, DC Red 27, DC Green 6, DC Yellow 11, DC Violet 2, DC Orange 5, Sudan red, carotenes (-carotene, lycopene), xanthophylls (capsanthin, capsorubin, lutein), palm oil, Sudan brown, quinoline yellow, annatto and curcumin.

The particulate dyestuffs may be present in a proportion of from 0.01% to 15% by weight relative to the total weight of the composition containing them.

They may especially be pigments, nacres and/or particles with metallic tints.

The term “pigments” should be understood as meaning white or coloured, mineral or organic particles that are insoluble in an aqueous solution, which are intended to colour and/or opacify the composition containing them.

Advantageously, a composition according to the invention may comprise from 0.01% to 25% by weight, especially from 0.1% to 25% by weight, in particular from 1% to 20% by weight and preferably from 5% to 15% by weight of pigments relative to the total weight of the said composition.

Preferably, when a composition according to the invention is a make-up composition, it may comprise at least 5%, and more preferably at least 10% by weight of pigments relative to the total weight of the said composition.

The pigments may be white or coloured, and mineral and/or organic.

As mineral pigments that may be used in the invention, mention may be made of titanium oxide, titanium dioxide, zirconium oxide, zirconium dioxide, cerium oxide or cerium dioxide and also zinc oxide, iron oxide or chromium oxide, ferric blue, manganese violet, ultramarine blue and chromium hydrate, and mixtures thereof.

It may also be a pigment having a structure that may be, for example, of sericite/brown iron oxide/titanium dioxide/silica type. Such a pigment is sold, for example, under the reference Coverleaf NS or JS by the company Chemicals and Catalysts, and has a contrast ratio in the region of 30.

They may also be pigments having a structure that may be, for example, of silica microsphere type containing iron oxide. An example of a pigment having this structure is the product sold by the company Miyoshi under the reference PC Ball PC-LL-100 P, this pigment being constituted of silica microspheres containing yellow iron oxide.

Advantageously, the pigments in accordance with the invention are iron oxides and/or titanium dioxides.

The term “nacres” should be understood as meaning iridescent or non-iridescent coloured particles of any shape, especially produced by certain molluscs in their shell or alternatively synthesized, which have a colour effect via optical interference.

A composition according to the invention may comprise from 0%/0 to 15% by weight of nacres relative to the total weight of the said composition.

The nacres may be chosen from nacreous pigments such as titanium mica coated with an iron oxide, titanium mica coated with bismuth oxychloride, titanium mica coated with chromium oxide, titanium mica coated with an organic dye and also nacreous pigments based on bismuth oxychloride. They may also be mica particles at the surface of which are superposed at least two successive layers of metal oxides and/or of organic dyestuffs.

Examples of nacres that may also be mentioned include natural mica coated with titanium oxide, with iron oxide, with natural pigment or with bismuth oxychloride.

Among the nacres available on the market, mention may be made of the nacres Timica, Flamenco and Duochrome (based on mica) sold by the company Engelhard, the Timiron nacres sold by the company Merck, the Prestige mica-based nacres sold by the company Eckart, and the Sunshine synthetic mica-based nacres sold by the company Sun Chemical.

The nacres may more particularly have a yellow, pink, red, bronze, orangey, brown, gold and/or coppery colour or tint.

Advantageously, the nacres in accordance with the invention are micas coated with titanium dioxide or with iron oxide, and also bismuth oxychloride.

For the purposes of the present invention, the term “particles with a metallic tint” means any compound whose nature, size, structure and surface finish allow it to reflect the incident light, especially in a non-iridescent manner.

The particles with a metallic tint that may be used in the invention are in particular chosen from:
particles of at least one metal and/or of at least one metal derivative;
particles comprising a single-material or multi-material organic or mineral substrate, at least partially coated with at least one layer with a metallic tint comprising at least one metal and/or at least one metal derivative; and
mixtures of the said particles.

Among the metals that may be present in the said particles, mention may be made, for example, of Ag, Au, Cu, Al, Ni, Sn, Mg, Cr, Mo, Ti, Zr, Pt, Va, Rb, W, Zn, Ge, Te and Se, and mixtures or alloys thereof. Ag, Au, Cu, Al, Zn, Ni, Mo and Cr, and mixtures or alloys thereof (for example bronzes and brasses) are preferred metals.

The term “metal derivatives” denotes compounds derived from metals, especially oxides, fluorides, chlorides and sulfides.

Illustrations of these particles that may be mentioned include aluminum particles, such as those sold under the names Starbrite 1200 EAC® by the company Siberline and Metalure® by the company Eckart and glass particles coated with a metallic layer, especially those described in documents JP-A-09188830, JP-A-10158450, JP-A-10158541, JP-A-07258460 and JP-A-05017710.

Hydrophobic Treatment of the Dyestuffs

The pulverulent dyestuffs as described previously may be totally or partially surface-treated, with a hydrophobic agent, to make them more compatible with the oily phase of the composition of the invention, especially so that they have good wettability with oils. Thus, these treated pigments are well dispersed in the oily phase.

Hydrophobic-treated pigments are described especially in document EP-A-1 086 683.

The hydrophobic-treatment agent may be chosen from silicones such as methicones, dimethicones and perfluoroalkylsilanes; fatty acids, for instance stearic acid; metal soaps, for instance aluminium dimyristate, the aluminium salt of hydrogenated tallow glutamate; perfluoroalkyl phosphates; polyhexafluoropropylene oxides; perfluoropolyethers; amino acids; N-acylamino acids or salts thereof; lecithin, isopropyl triisostearyl titanate, isostearyl sebacate, and mixtures thereof.

The term “alkyl” mentioned in the compounds cited above especially denotes an alkyl group containing from 1 to 30 carbon atoms and preferably containing from 5 to 16 carbon atoms.

### Fillers

For the purposes of the present invention, the term “fillers” should be understood as meaning colourless or white solid particles of any form, which are in an insoluble and dispersed form in the medium of the composition.

These fillers, of mineral or organic, natural or synthetic nature, give the composition containing them softness and give the makeup result a matt effect and uniformity.

Preferably, a composition of the invention comprises fillers, particularly when it is dedicated to provide a high coverage.

In particular, a composition according to the invention may comprise from 2% to 35% by weight, especially from 5% to 35% by weight, in particular from 5% to 20% by weight.

According to one embodiment of the invention, a composition may comprise solid particles such as pigments and/or fillers.

Advantageously, a composition according to the invention may comprise from 0.01% to 25% by weight, especially from 0.1% to 25% by weight, in particular from 1% to 20% by weight and preferably from 5% to 15% by weight of solid particles relative to the total weight of the said composition.

Preferably, when a composition according to the invention is a make-up composition, it may comprise at least 5%, and more preferably at least 10% by weight of solid particles relative to the total weight of the said composition.

### Dispersant

Advantageously, a composition according to the invention may also comprise a dispersant.

Such a dispersant may be a surfactant, an oligomer, a polymer or a mixture of several thereof.

According to one particular embodiment, a dispersant in accordance with the invention is a surfactant.

### Active Agent

For a particular care application, a composition according to the invention may comprise at least one moisturizer (also known as a humectant).

Preferably, such moisturizer is glycerol.

The moisturizer(s) could be present in the composition in a content ranging from 0.1% to 15% by weight, especially from 0.5% to 10% by weight or even from 1% to 6% by weight, relative to the total weight of the said composition.

As other active agents that may be used in the composition of the invention, examples that may be mentioned include vitamins and sunscreens, and mixtures thereof.

Preferably, a composition of the invention comprises at least one active agent.

It is a matter of routine for those skilled in the art to adjust the nature and amount of the additives present in the compositions in accordance with the invention such that the desired cosmetic properties thereof are not thereby affected.

According to one embodiment, a composition of the invention may advantageously be in the form of a foundation.

According to one embodiment, a composition of the invention may advantageously be in the form of a composition for making up the skin and especially the face. It may thus be an eyeshadow or a face powder.

According to another embodiment, a composition of the invention may advantageously be in the form of a lip product, and in particular a lipstick.

According to another embodiment, a composition of the invention may advantageously be in the form of a composition for caring for the skin of the body or the face, in particular the face.

According to another embodiment, a composition of the invention may be in the form of a product for the eyelashes, in particular a mascara.

Such compositions are especially prepared according to the general knowledge of a person skilled in the art.

Throughout the description, including the claims, the term “comprising a” should be understood as being synonymous with “comprising at least one”, unless otherwise specified.

The expressions “between . . . and . . . ” and “ranging from . . . to . . . ” should be understood as meaning limits included, unless otherwise specified.

The invention is illustrated in greater detail by the examples and FIGURES presented below. Unless otherwise mentioned, the amounts indicated are expressed as weight percentages.

Methodology for the Oscillating Dynamic Rheology Measurements

These are rheological measurements in the harmonic regime, which measure the elastic modulus.

The measurements are taken using a Haake RS600 rheometer on a product at rest, at 25° C. with a plate-plate rotor Ø 60 mm and a 2 mm gap.

The measurements in the harmonic regime make it possible to characterize the viscoelastic properties of the products. The technique consists in subjecting a material to a stress that varies sinusoidally over time and in measuring the response of the material to this stress. In a region in which the behaviour is linearly viscoelastic (zone in which the strain is proportional to the stress), the stress (τ) and the strain (γ) are two sinusoidal functions of time that are written in the following manner:

τ(t)=τ₀ sin(ωt)

γ(t)=γ₀ sin(ωt+δ)

in which:

τ₀ represents the maximum amplitude of the stress (Pa);

γ₀ represents the maximum amplitude of the strain (−);

ω=2ΠN represents the angular frequency (rad·s⁻¹) with N representing the frequency (Hz); and

δ represents the phase angle of the stress relative to the strain (rad).

Thus, the two functions have the same angular frequency, but they are dephased by an angle δ. According to the phase angle δ between τ(t) and γ(t), the behaviour of the system may be assessed:
if δ=0, the material is purely elastic;
if δ=11/2, the material is purely viscous (Newtonian fluid); and
if 0<δ<Π/2, the material is viscoelastic.

In general, the stress and the strain are written in complex form:

τ*(t)=τ₀e^{iωt}

γ*(t)=γ₀e^{(iωt+δ)}

A complex stiffness modulus, representing the overall resistance of the material to the strain, whether it is of elastic or viscous origin, is then defined by:

G*=τ*/γ*=G′+iG″

in which:

G′ is the storage modulus or elastic modulus, which characterizes the energy stored and totally restituted in the course of a cycle, G′=(τ₀/γ₀) cos δ; and

G″ is the loss modulus or viscous modulus, which characterizes the energy dissipated by internal friction in the course of a cycle, G″=(τ/γ₀) sin δ.

The parameter retained is the mean stiffness modulus G* recorded at the plateau measured at a frequency of 1 Hz.

### EXAMPLE 1

Foundation formulations in accordance with the invention are prepared from the phases described below.
1) Preparation of the aqueous phase A1

The aqueous phase is prepared from the compounds that follow in the weight proportions stated in the table below.

Phase A1:

Compounds Weight %

| | Weight % |
|---|---|
| Compounds | Phase A1 |
| Water | qs 100 |
| Glycerol | 10.00 |
| Butylene glycol | 6.25 |
| Phenoxyethanol | 0.63 |
| Caprylyl glycol | 0.63 |
| Hydroxyethyl acrylate/ | 1.74 |
| sodium acryloyldimethyltaurate copolymer | |
| (Sepinov ^{®} EMT 10 sold by the company | |
| SEPPIC) | |

The water, glycerol, butylene glycol, phenoxyethanol and caprylyl glycol are weighed out in a beaker and stirred using a Rayneri blender at room temperature.

The hydroxyethyl acrylate/sodium acryloyldimethyltaurate copolymer is added with stirring at room temperature. The stirring is adjusted so as not to incorporate air into the mixture.

The mixture is stirred moderately for about 10 minutes at room temperature.

2) Preparation of the Oily Phases

The oily phases are prepared from the compounds that follow in the weight proportions stated in the tables below.

Phase B1:

| | Weight % |
|---|---|
| Compounds | Phase B1 |
| Yellow iron oxide | 4.92 |
| Red iron oxide | 1.00 |
| Black iron oxide | 0.35 |
| Titanium dioxide | 31.24 |
| Isononyl isononanoate | qs 100 |
| Ethylenediamine/stearyl dimer dilinoleate | 12 |
| copolymer (Uniclear ^{®} 100 VG sold by the | |
| company Arizona Chemical) | |

The pigments are ground with 15% of the oils using a three-roll mil.

The ground material, the remainder of the oils and the ethylenediamine/stearyl dimer dilinoleate copolymer are placed in a beaker and stirred using a Rayneri blender.

The mixture is heated to 100° C. until dissolution of the ethylenediamine/stearyl dimer dilinoleate copolymer is complete (about 20 to 25 minutes).

The mixture is allowed to cool to room temperature.

Phase B2:

| | Weight % |
|---|---|
| Compounds | Phase B2 |
| Yellow iron oxide | 4.92 |
| Red iron oxide | 1.00 |
| Black iron oxide | 0.35 |
| Titanium dioxide | 31.24 |
| Isononyl isononanoate | qs 100 |
| Nylon-611/dimethicone copolymer (Dow | 11.00 |
| Corning 2-8179 Gellant sold by the | |
| company Dow Corning) | |

The pigments are ground with 15% of the oils using a three-roll mill.

The ground material, the remainder of the oils and the Nylon-611/dimethicone copolymer are placed in a beaker and stirred using a Rayneri blender.

The mixture is heated to 100° C. until dissolution of the Nylon-611/dimethicone copolymer is complete (about 20 to 25 minutes).

The mixture is allowed to cool to room temperature.

3) Preparation of Foundation Formulations

These formulations are obtained by mixing several phases intended to form the foundations in accordance with the invention, in the proportions described below in Table 1.

The aqueous and oily phases are weighed out and then stirred moderately for about 10 minutes.

The combination of the various phases is established as a function of the desired properties.

TABLE I

| | Technical | Weight | Weight | Weight |
|---|---|---|---|---|
| | performance | % | % | % |
| | quality(ies) | Phase | Phase | Phase |
| Formulations | obtained | A1 | B1 | B2 |
| Formulation | Freshness and | 60.00 | 40.00 | |
| 1 | radiance/ | | | |
| | luminous | | | |
| Formulation | Freshness and | 60.00 | | 40.00 |
| 2 | radiance/ | | | |
| | luminous | | | |

Formulation 1 is thick and has a foam appearance. The formulation spreads well, and the deposit gives average coverage and is homogeneous. The skin finish is satiny and slightly tacky.

Formulation 2 is creamy and has a foam appearance. The formulation spreads very well and gives a satiny/glossy deposit, which is homogeneous and slightly tacky.

1. Cosmetic gel-gel type composition for making up and/or caring for keratin materials, comprising:
at least one aqueous phase gelled with at least one hydrophilic gelling agent selected from the group consisting of copolymers of (1) 2-acrylamido-2-methylpropanesulfonic acid and (2) hydroxyethyl acrylate; and
at least one oily phase gelled with at least one lipophilic gelling agent selected from the group consisting of hydrocarbon-based polyamides, silicone polyamides, and mixtures thereof,
wherein either
(a) the at least one hydrophilic gelling agent is a copolymer of 2-acrylamido-2-methylpropanesulfonic acid and of hydroxyethyl acrylate, and the at least one lipophilic gelling agent is ethylenediamine/stearyl dimer dilinoleate copolymer, or
(b) the at least one hydrophilic gelling agent is a copolymer of 2-acrylamido-2-methylpropanesulfonic acid and of hydroxyethyl acrylate, and the at least one lipophilic gelling agent is Nylon-611/dimethicone copolymer.

2. The composition according to claim 1, containing at least one dyestuff.

3. The composition according to claim 1, containing the aqueous and oily phases in an aqueous phase/oily phase weight ratio of from 95/5 to 5/95.

4. The composition according to claim 1, which is in the form of a foundation, a face powder, an eyeshadow, a lipstick, a mascara and/or a care composition.

5. The composition according to claim 1, further comprising solid particles.

6. The composition according to claim 5, comprising from 0.01% to 25% by weight of solid particles relative to the total weight of the said composition.

7. The composition according to claim 1, further comprising volatile and/or non volatile silicone oils.

8. The composition according to claim 1, further comprising a moisturizer.

9. Process for preparing a cosmetic composition for making up and/or caring for keratin materials, comprising at least one step of mixing:
at least one aqueous phase gelled with at least one hydrophilic gelling agent selected from the group consisting of synthetic polymeric gelling agents; and
at least one oily phase gelled with at least one hydrogen bonding polymer selected from the group consisting of hydrocarbon-based polyamides, silicone polyamides, and mixtures thereof;
under conditions suitable for obtaining a macroscopically homogeneous mixture,
the cosmetic composition not comprising a sunscreen.

10. The process according to claim 9, comprising a step of mixing at least three or even more gelled phases.

11. The process according to claim 9, in which the mixing is performed at room temperature.

12. Cosmetic kit for making up and/or caring for keratin materials, comprising, in separate containers,
at least one aqueous phase gelled with at least one hydrophilic gelling agent selected from the group consisting of synthetic polymeric gelling agents, and
at least one oily phase gelled with at least one hydrogen bonding polymer selected from the group consisting of hydrocarbon-based polyamides, silicone polyamides, and mixtures thereof, and also
instructions for using the extemporaneous mixtures,
the at least one aqueous phase and at least one oily phase not comprising a sunscreen.

13. Device for making up and/or caring for keratin materials, comprising at least:
two separate containers containing, respectively, an aqueous phase gelled with at least one hydrophilic gelling agent selected from the group consisting of synthetic polymeric gelling agents, and an oily phase gelled with at least one hydrogen bonding polymer selected from the group consisting of hydrocarbon-based polyamides, silicone polyamides, and mixtures thereof, the aqueous phase and oily phase not comprising a sunscreen;
a distinct chamber for mixing the said containers, comprising an aperture configured to allow the introduction of the said phases to be mixed; and
a means for distributing a macroscopically homogeneous mixture of the two phases.

14. Cosmetic process for making up and/or caring for keratin materials, comprising at least one step which consists in applying to the said keratin material a cosmetic composition for making up and/or caring for keratin materials, comprising at least one aqueous phase gelled with at least one hydrophilic gelling agent selected from the group consisting of synthetic polymeric gelling agents; and at least one oily phase gelled with at least one hydrogen bonding polymer selected from the group consisting of hydrocarbon-based polyamides, silicone polyamides, and mixtures thereof; the said phases forming therein a macroscopically homogeneous mixture, the cosmetic composition not comprising a sunscreen.

15. Cosmetic process for making up and/or caring for a keratin material, comprising at least the application to the said material of a composition obtained by extemporaneous mixing, before application or at the time of application to the said keratin material, of at least one aqueous phase gelled with at least one hydrophilic gelling agent selected from the group consisting of synthetic polymeric gelling agents, and at least one oily phase gelled with at least one hydrogen bonding polymer selected from the group consisting of hydrocarbon-based polyamides, silicone polyamides, and mixtures thereof, the composition not comprising a sunscreen.

16. The composition according to claim 1, for making up and/or caring for the skin and/or the lips.

17. The composition according to claim 1, containing at least one dyestuff present at least in the gelled oily phase.

18. The composition according to claim 1, containing the aqueous and oily phases in an aqueous phase/oily phase weight ratio of from 60/40 to 70/30.

19. The composition according to claim 1, wherein the at least one hydrophilic gelling agent is a copolymer of 2-acrylamido-2-methylpropanesulfonic acid and of hydroxyethyl acrylate, and the at least one lipophilic gelling agent is ethylenediamine/stearyl dimer dilinoleate copolymer.

20. The composition according to claim 1, wherein the at least one hydrophilic gelling agent is a copolymer of 2-acrylamido-2-methylpropanesulfonic acid and of hydroxyethyl acrylate, and the at least one lipophilic gelling agent is Nylon-611/dimethicone copolymer.

21. The composition according to claim 1, wherein the at least one lipophilic gelling agent is present in an amount of between 0.1% and 30% by weight, relative to a total weight of the at least one oily phase.

22. The composition according to claim 2, wherein the at least one dyestuff is selected from pigments, nacres, particles with metallic tints, and mixtures thereof.

23. The composition according to claim 21, wherein the moisturizer is glycerol.

24. The composition according to claim 21, wherein the moisturizer is present in the composition in a content from 0.1% to 15% by weight relative to the total weight of the composition.
